(19) United States
(12) Patent Application Publication
(10) Pub. No.: US 2010/0048722 A1
(43) Pub. Date: Feb. 25, 2010

(54) FORMATION OF ACCUMBENS GluR2-LACKING AMPA RECEPTORS MEDIATES INCUBATION OF COCAINE CRAVING

(75) Inventors: Marina E. WOLF, Libertyville, IL (US); Michela Marinelli, Chicago, IL (US); Kuei Y. Tseng, Chicago, IL (US); Kelly L. Conrad, Goodlettsville, TN (US)

Correspondence Address: Joseph A. Fuchs;Rockey, Depke & Lyons, LLC, 233 S. Wacker Drive, Suite 5450, Sears Tower, Chicago, IL, 60606-6306, US

(73) Assignee: ROSALIND FRANKLIN UNIVERSITY OF MEDICINE AND SCIENCE, North Chicago, IL (US)

(21) Appl. No.: 12/471,140

(22) Filed: May 22, 2009

Related U.S. Application Data
(60) Provisional application No. 61/128,524, filed on May 22, 2008.

(51) Int. Cl. A61K 31/165; A61P 25/30

(52) U.S. Cl. 514/626

(57) ABSTRACT

The present invention provides a method for ameliorating cue-induced cravings for an addictive substance in abstinent addicts by administering a compound capable of blockade of GluR2-lacking AMPA receptors.

### RELATED APPLICATION

This application claims priority to U.S. Provisional Patent Application No. 61/128,524, filed on May 22, 2008, which is incorporated herein in its entirety by reference and made a part hereof.

### BACKGROUND OF THE INVENTION

1. Technical Field

The present invention is generally related to a treatment strategy, based on blockade of GluR2-lacking AMPA receptors, to decrease cue-induced cocaine craving in abstinent cocaine addicts.

2. Background Art

Relapse to cocaine use after prolonged abstinence is a major clinical problem. This relapse is often induced by exposure to cues associated with cocaine use. To account for the persistent propensity for relapse, Gawin and Kleber¹ suggested that cue-induced cocaine craving increases over the first several weeks of abstinence and remains high for extended periods. We and others identified an analogous phenomenon in rats that was termed “incubation of cocaine craving”: time-dependent increases in cue-induced cocaine-seeking over the first months after withdrawal from self-administered cocaine²⁻⁴. Cocaine-seeking requires activation of glutamate projections that excite AMPA receptors in the nucleus accumbens⁵⁻⁷. Here we demonstrate that the number of synaptic AMPA receptors in the accumbens is increased after prolonged withdrawal from cocaine self-administration by the addition of new GluR2-lacking AMPA receptors. Furthermore, we show that these new receptors mediate the incubation of cocaine craving. Our results suggest GluR2-lacking AMPA receptors as a novel target for drug development for the treatment of cocaine addiction. We propose that after prolonged withdrawal from cocaine, increased synaptic AMPA receptor number combined with the higher conductance of GluR2-lacking AMPA receptors^{8,9} causes increased reactivity of accumbens neurons to cocaine-related cues, leading to intensification of drug craving and relapse.

### BRIEF DESCRIPTION OF THE DRAWINGS

FIG. 1 Time-dependent increases in cue-induced cocaine-seeking (incubation of cocaine craving). (a) Experimental timeline. (b) Training: Mean±S.E.M number of infusions (each paired with 5-sec light-cue) during training. Cocaine (0.5 mg/kg/infusion) supported self-administration as indicated by high nose-poking in the active hole. Responding in the inactive hole was very low (not shown). (c) Drug-seeking tests: Number of nose-pokes in the previously active hole (a measure of cocaine-seeking) and inactive hole during a 30-min test performed under extinction conditions (nose-pokes deliver cue but not cocaine). Cocaine-seeking increased on withdrawal day 45 (withdrawal day X hole interaction: F_{1,12}=14.9, p<0.01) (n=7/group). *Different from withdrawal day 1, p<0.05.

FIG. 2 GluR1 and GluR3 increase after withdrawal from cocaine self-administration. (a-d) GluR1 increased dramatically in cocaine-exposed rats on withdrawal day 45 (surface, intracellular, and total GluR1: drug exposure X day interaction, F_{1,56}=9.9, F_{1,56}=9.2, and F_{1,56}=12.1, p<0.01). (e-h) GluR2 was unchanged by cocaine self-administration except for a small increase in the surface/intracellular ratio on withdrawal day 1 (drug exposure X day interaction, F_{1,53}=4.0, p<0.01). (i-l) Surface GluR3 and the surface/intracellular ratio increased after cocaine self-administration (drug exposure, F_{1,48}=4.4, and F_{1,48}=3.9, p<0.05). Data (mean±S.E.M, n=12-18/group) expressed as percentage of day 1 saline. *Different from other conditions, p<0.05; ^{#}Different from saline day 1, p<0.05.

FIG. 3. GluR2-lacking AMPA receptors are detected in accumbens neurons after prolonged withdrawal from cocaine self-administration. (a) Evoked EPSC recorded after 42-47 withdrawal days. (b) Current-voltage relationships for neurons shown in a. (c) Rectification index (EPSC_{-70 mV}/EPSC_{+40 mV}; FIGS. 5-12) from 13 and 8 neurons recorded from 4 cocaine and 3 saline rats (t₁₉=3.47, *p<0.01). (d-e) Naspm (200 μM, 5-10 min) reduced evoked EPSC amplitude in cocaine-exposed rats (t₆=4.72, *p<0.01, baseline vs. Naspm, 7 cells/group). Naspm effect illustrated as evoked EPSC amplitude normalized to baseline (t₁₂=3.73, *p<0.01). (g) Representative traces illustrating Naspm effect after 10 min of bath application. (h) Location of recordings. B: Bregma.

FIG. 4 Enhanced cue-induced cocaine-seeking after prolonged withdrawal from cocaine self-administration is inhibited by blockade of GluR2-lacking AMPA receptors. (a) Left: Responses (mean±S.E.M) on previously active or inactive levers after Naspm or vehicle injections into accumbens 15 min before extinction tests on withdrawal days 1 or 45 (n=10-14/group). Right: Responses on previously active lever at each hour of test (Naspm-dose X withdrawal-day X session-hour X lever interaction: F_{1,45}=4.6, p<0.05). (b) Naspm accumbens injections had no effect on sucrose or cocaine self-administration. Mean±S.E.M number of oral sucrose (0.75 mL/delivery, n=10) or intravenous cocaine (0.75 mg/kg/infusion, n=5) deliveries. (c) Injector tips. *Different from other groups, p<0.05.

FIG. 5. Cocaine-exposed rats at intermediate withdrawal times (3 and 21 days) show intermediate changes in GluR1 expression and distribution.

FIG. 6 Quantitative co-immunoprecipitation of AMPA receptor subunits in the nucleus accumbens after prolonged withdrawal from cocaine self-administration.

FIG. 7 Effect of cocaine self-administration and subsequent withdrawal on AMPA receptor subunit expression and distribution in the ventral tegmental area (VTA).

FIG. 8 Cocaine self-administration and subsequent prolonged withdrawal does not significantly alter AMPA receptor subunit expression and distribution in the cingulate cortex.

FIG. 9 Cocaine self-administration and subsequent prolonged withdrawal does not significantly alter NMDA receptor subunit expression and distribution in the nucleus accumbens.

FIG. 10 AMPA receptor adaptations after withdrawal from cocaine self-administration occur primarily in the nucleus accumbens core subregion.

FIG. 11 A 30 min cue-induced cocaine-seeking test has little effect on AMPA receptor expression and redistribution in the nucleus accumbens after withdrawal from cocaine self-administration.

FIG. 12 Nucleus accumbens medium spiny neurons recorded after prolonged withdrawal from cocaine self-administration exhibit increased frequency and amplitude of spontaneous EPSCs (sEPSC) compared with the saline-exposed group but no change in the paired-pulse facilitation ratio.

### DETAILED DESCRIPTION OF THE INVENTION

While this invention is susceptible of embodiment in many different forms, there is shown in the drawings, and will be described herein in detail, specific embodiments thereof with the understanding that the present disclosure is to be considered as an exemplification of the principles of the invention and is not intended to limit the invention to the specific embodiments illustrated.

The present invention provides a method for ameliorating cue-induced cravings for substances, including substances capable of forming psychological and physiological addictions. The substances can include those having therapeutic effects, pharmacological activity, illicit drugs, alcohol, nicotine, caffeine, and food. The method includes administering to abstinent users of such substances a compound capable of forming a blockade of GluR2-lacking AMPA receptors

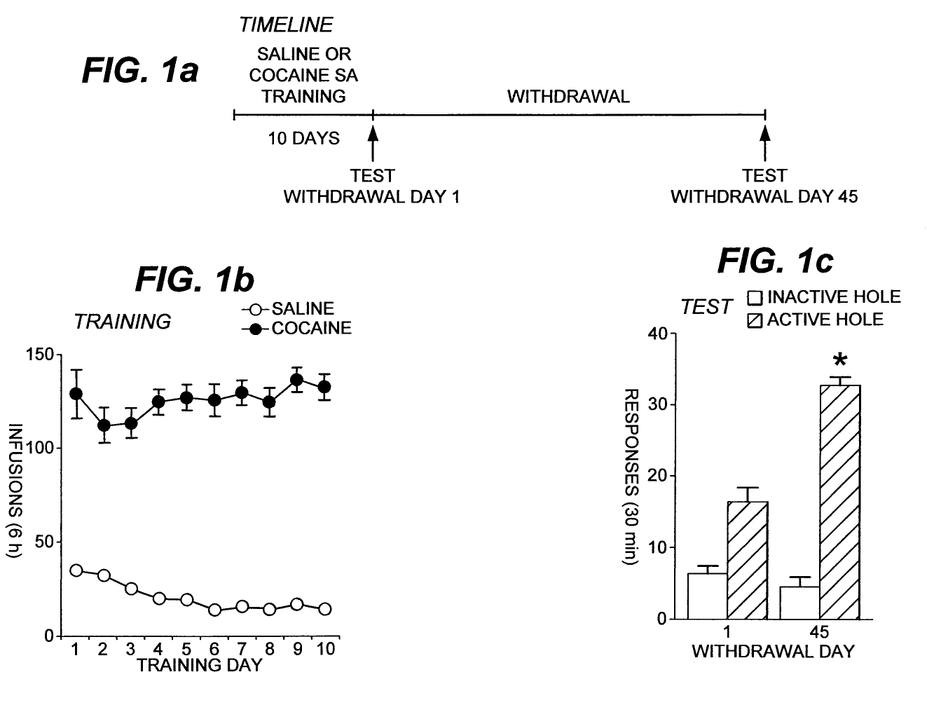

We trained rats for 6 h/day for 10 days to nose-poke in order to receive intravenous cocaine or saline infusions (FIGS. 1a, b); these infusions were paired with a 5-sec light cue. After 1 or 45 days of withdrawal from cocaine or saline self-administration, we assessed cue-induced cocaine-seeking in 30 min extinction tests. In these tests, rats were exposed to cues previously associated with cocaine availability but nose-poke responding in the previously active hole (a measure of cocaine-seeking) did not result in cocaine infusions. Consistent with prior results^{2,3} cue-induced cocaine-seeking was significantly greater on withdrawal day 45 than on withdrawal day 1 (FIG. 1c), confirming that cocaine craving incubates over time.

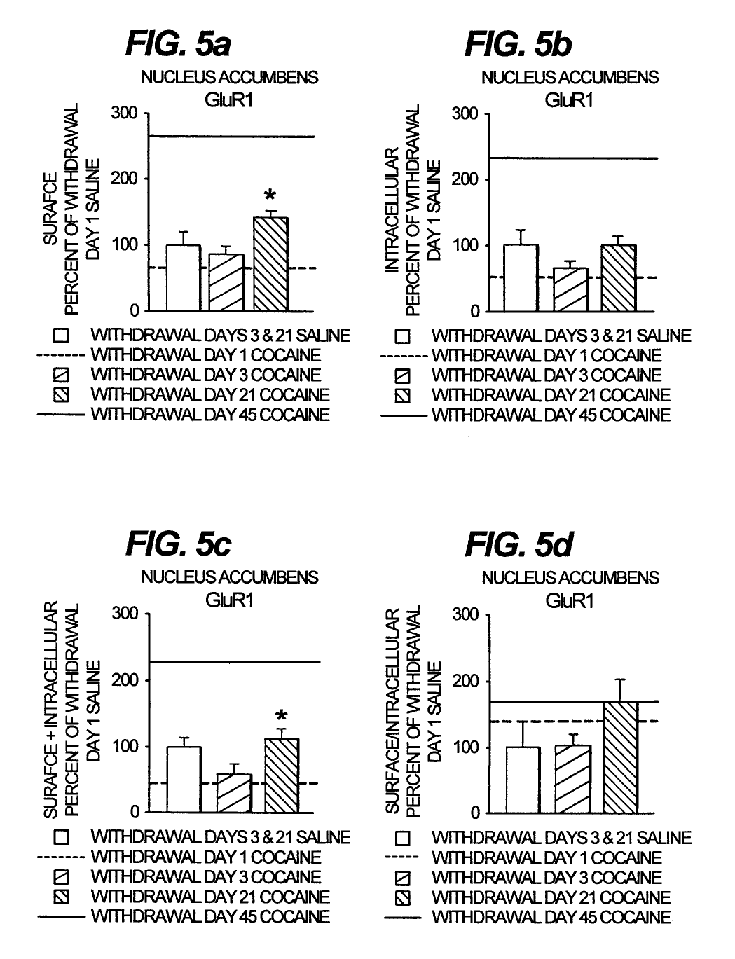

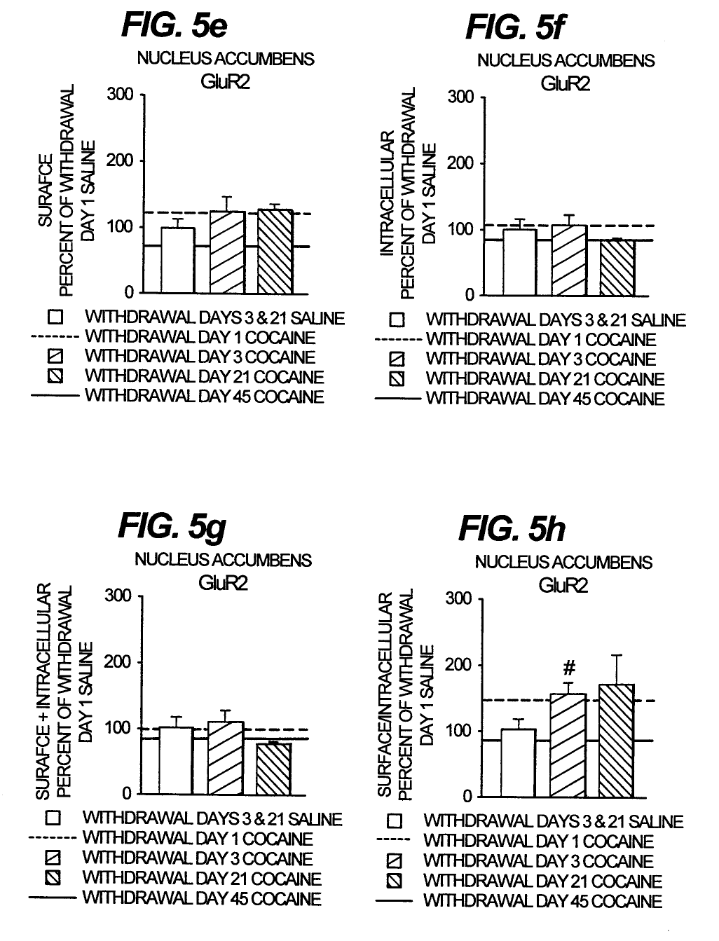

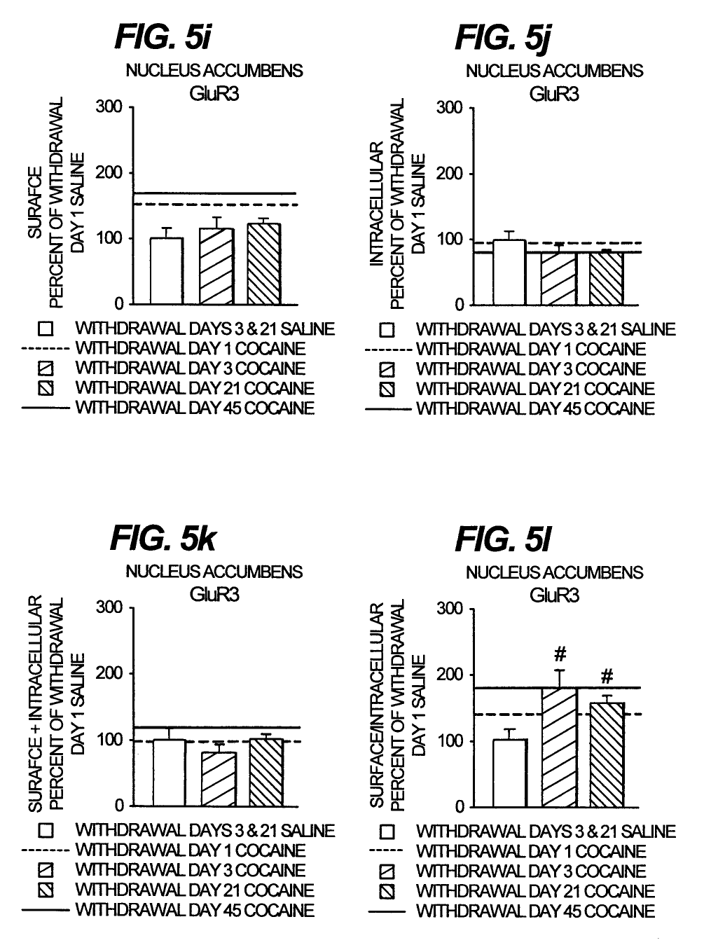

Based on a critical role for glutamate-dependent plasticity in other addiction models¹⁰⁻¹², and our previous work^{13,14}, we hypothesized that time-dependent increases in accumbens AMPA receptor transmission underlie the incubation of cocaine craving. To test this hypothesis, we trained rats to self-administer cocaine or saline (as described above) for subsequent biochemical analysis after 1 or 45 withdrawal days. The experimental groups were withdrawal day 1-saline (WD1-SAL), withdrawal day 1-cocaine (WD1-COC), withdrawal day 45-saline (WD45-SAL), and withdrawal day 45-cocaine (WD45-COC). We determined AMPA receptor distribution with a BS³ protein crosslinking assay that enables quantification of surface and intracellular receptor pools in tissue harvested after in vivo treatments^{13,14} (FIGS. 5-12).

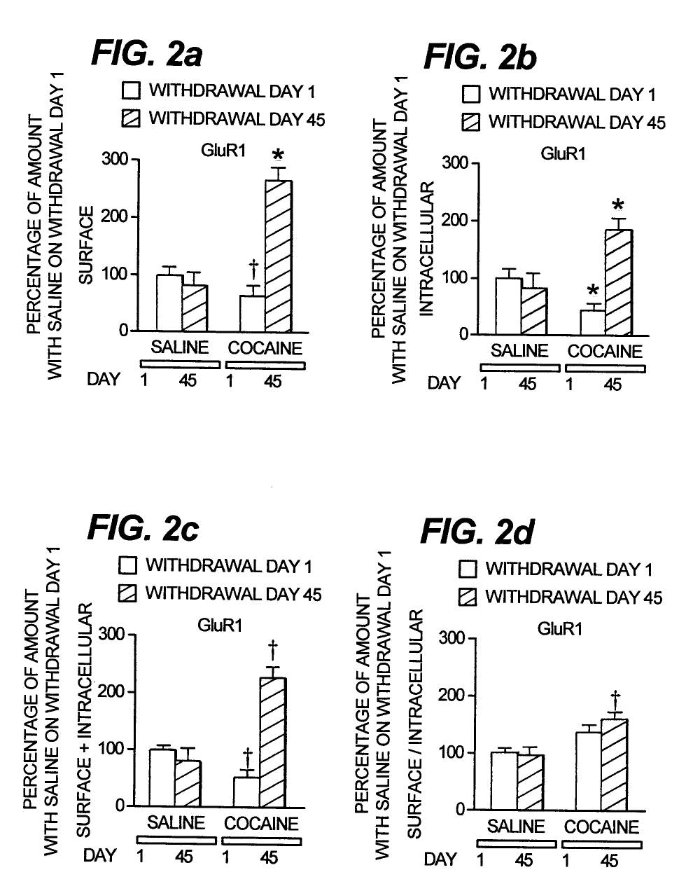

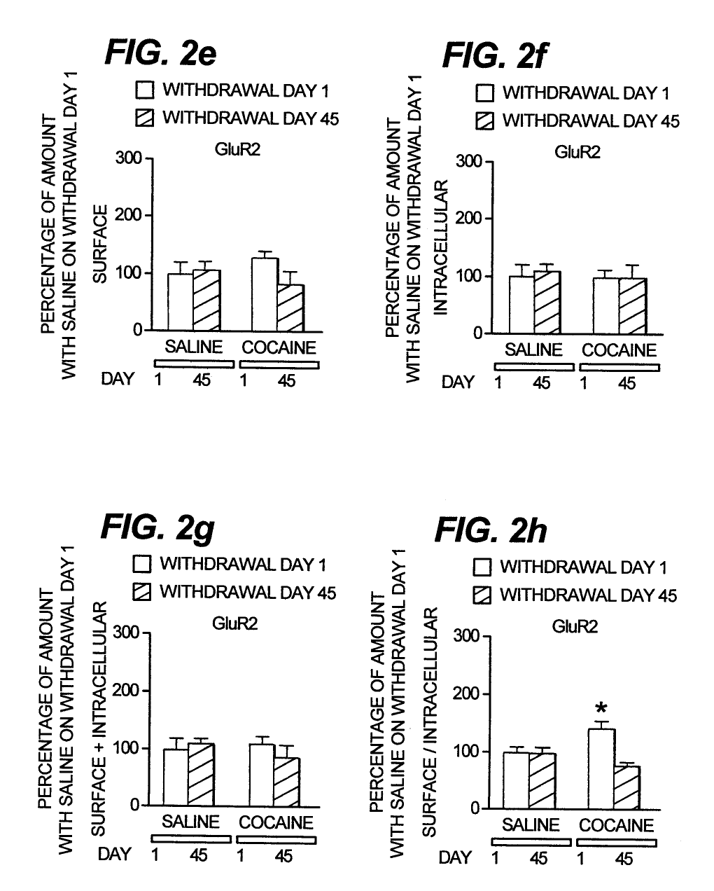

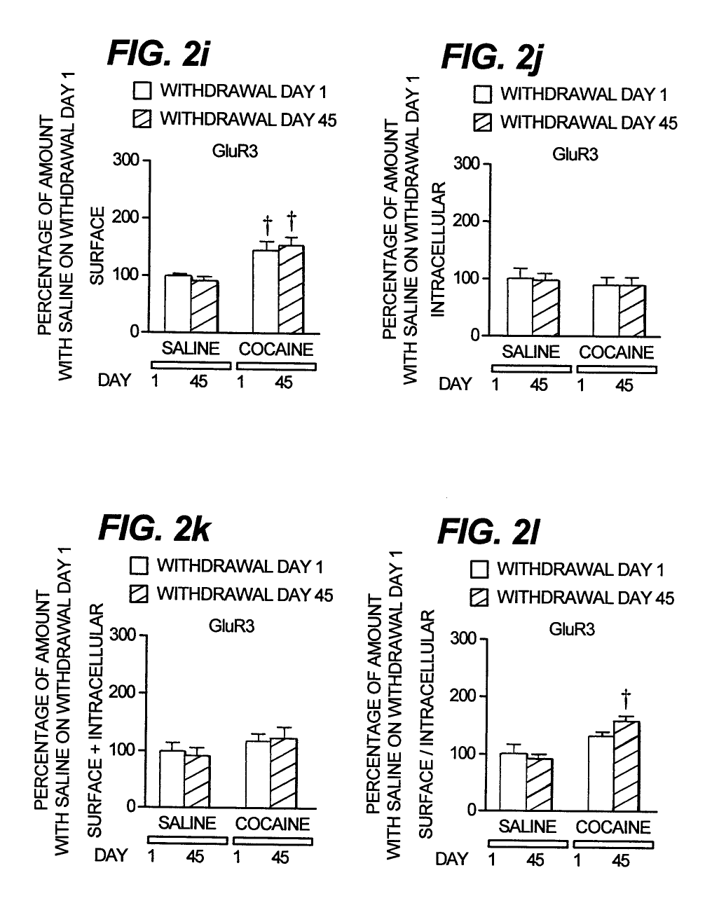

We found substantial (over 2-fold) increases in surface, intracellular and total GluR1 levels in the WD45-COC group compared with all other groups, as well as a more modest increase in the GluR1 surface/intracellular ratio (FIG. 2d). Thus, the major effect of prolonged withdrawal from cocaine is increased GluR1 expression, rather than redistribution of pre-existing GluR1, suggesting either increased GluR1 synthesis or reduced GluR1 degradation. The magnitude of increased GluR1 expression indicates a locus in medium spiny neurons, which comprise over 90% of accumbens neurons¹⁵. We found opposite changes in GluR1 on withdrawal day 1: rats in the WD1-COC group had significantly lower surface, intracellular, and total GluR1 levels (FIG. 2a-c). Intermediate effects were observed in an additional experiment that examined withdrawal days 3 and 21 (FIG. 5), indicating that GluR1 levels increase gradually after withdrawal from cocaine. For GluR2, we found a small increase in the surface/intracellular ratio on withdrawal day 1, but no changes on withdrawal day 45 (FIG. 2h). For GluR3, we found increased surface expression on both withdrawal days 1 and 45, indicating a time-independent effect (FIG. 2i).

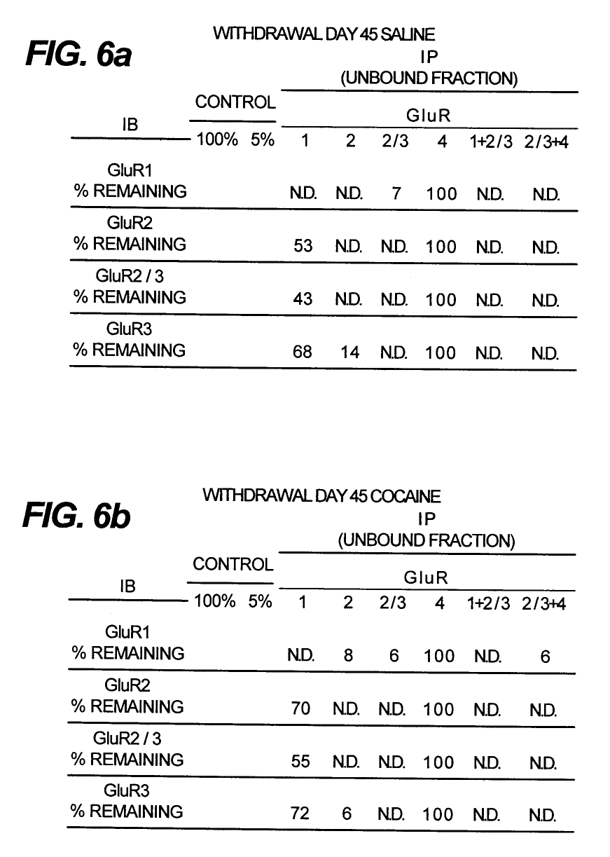

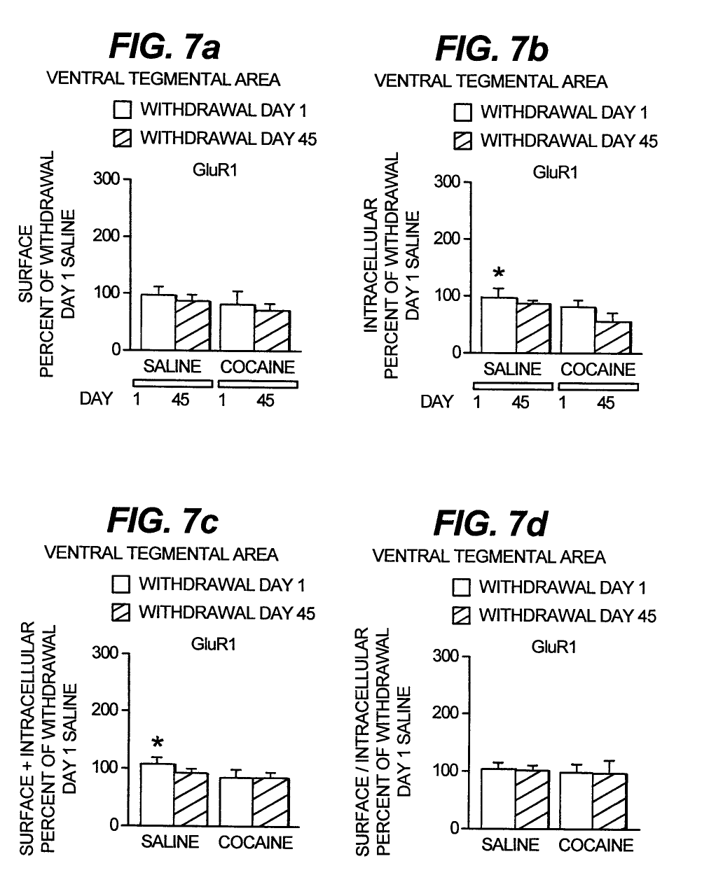

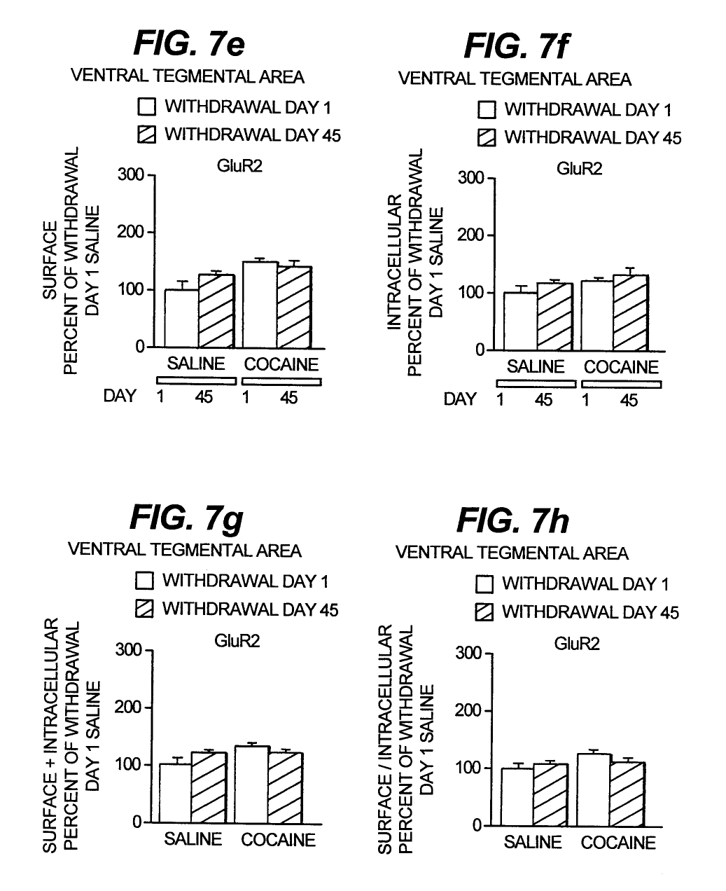

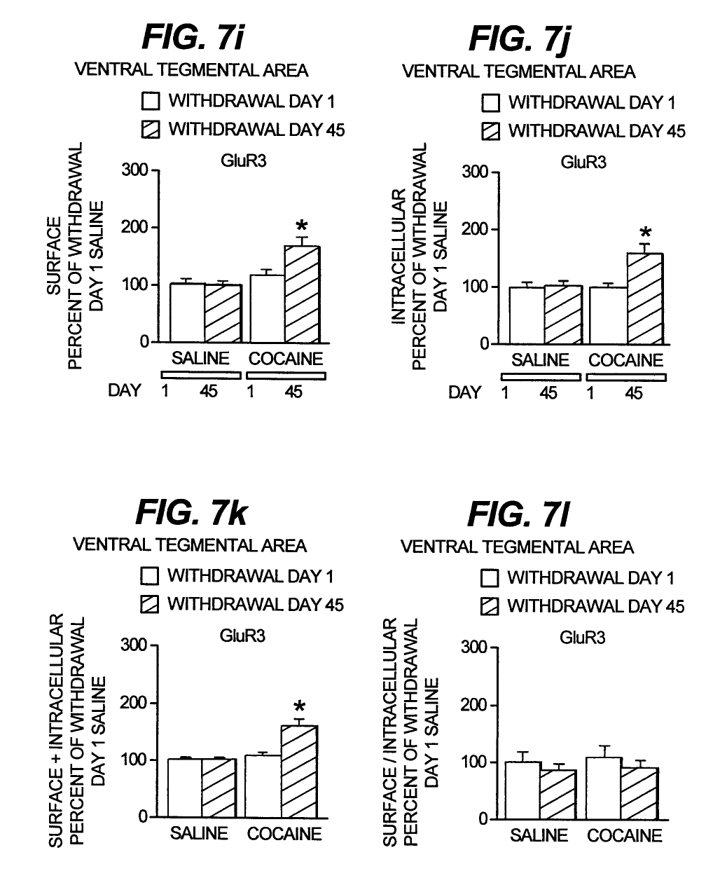

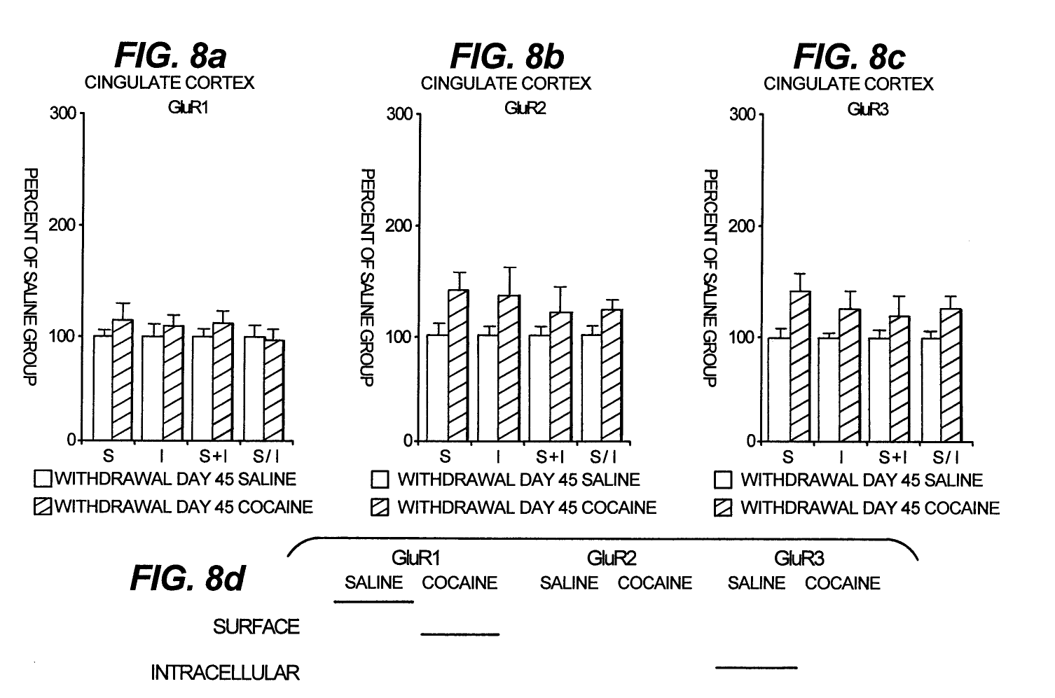

These results suggest that after prolonged withdrawal from cocaine, the normal complement of GluR2-containing AMPA receptors is supplemented by the addition of GluR2-lacking receptors (GluR1/3 and/or homomeric GluR1). We obtained additional support for this conclusion from a quantitative co-immunoprecipitation experiment (FIG. 6). This effect is specific to accumbens AMPA receptors: we found no evidence for the formation of GluR2-lacking AMPA receptors in ventral tegmental area (VTA) or cingulate cortex after withdrawal from cocaine (FIGS. 7, 8). Nor did we find significant changes in accumbens NMDA receptor subunits (FIG. 8).

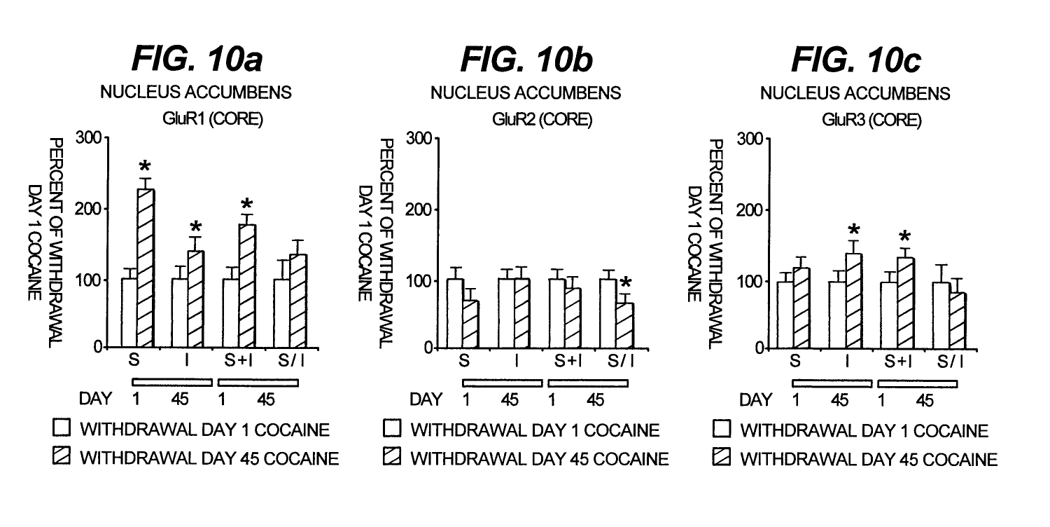

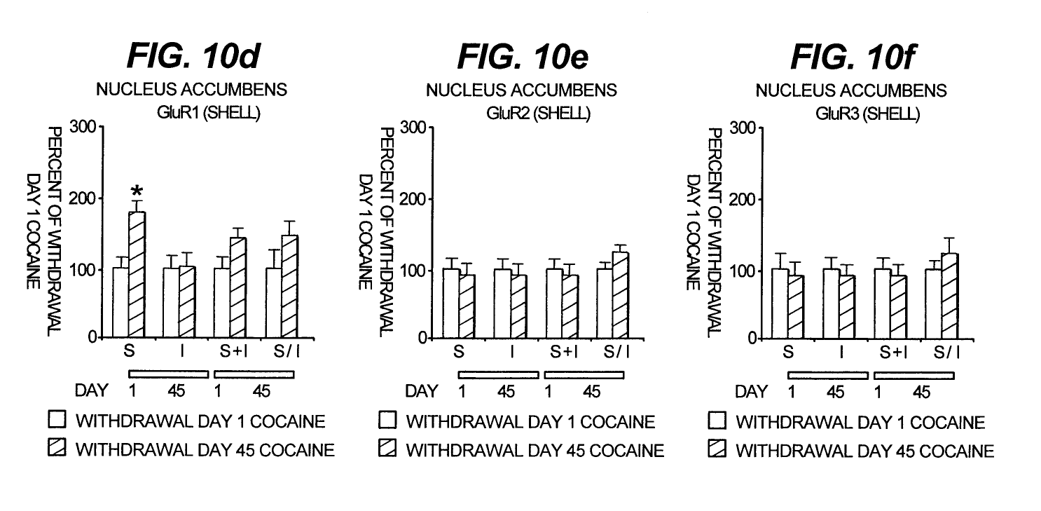

The accumbens consists of two major subregions, termed core and shell, which can be distinguished based on connectivity and morphology¹⁵. The core and shell play different roles in drug-related behaviors, with some evidence suggesting that the core plays a more significant role in cue-induced cocaine-seeking¹⁶. To study potential core-shell differences, we assessed another cohort of cocaine self-administering rats after 1 or 45 withdrawal days. We divided the accumbens into core and shell subregions, crosslinked with BS³, and analyzed GluR1-3 (FIG. 10). In the core, we found robust time-dependent increases in GluR1 and modest increases in GluR3. In the shell, we found that surface GluR1 was increased on withdrawal day 45. These results suggest GluR2-lacking AMPA receptors form in both core and shell, but this effect may be more pronounced in the core.

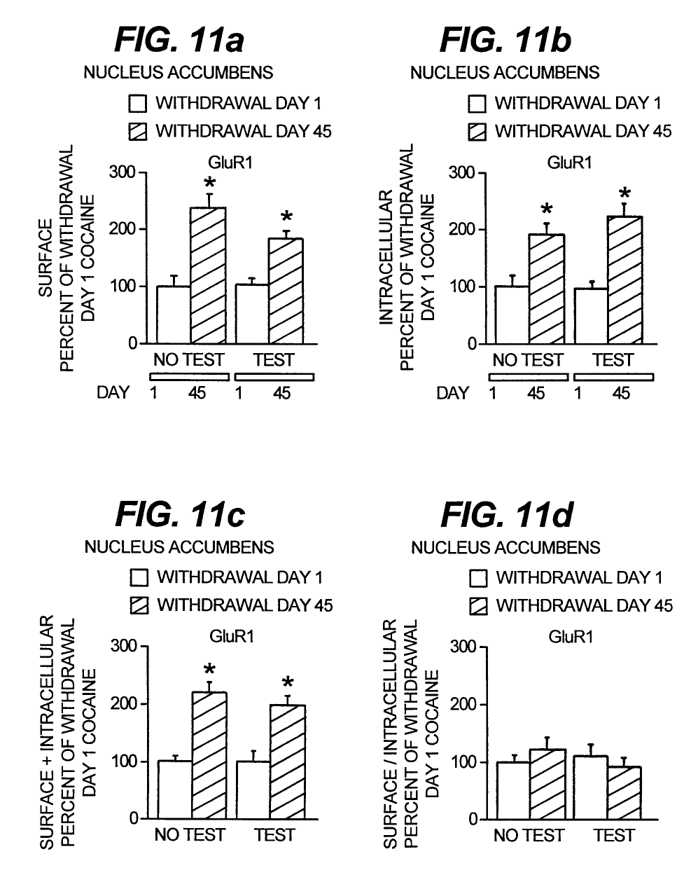

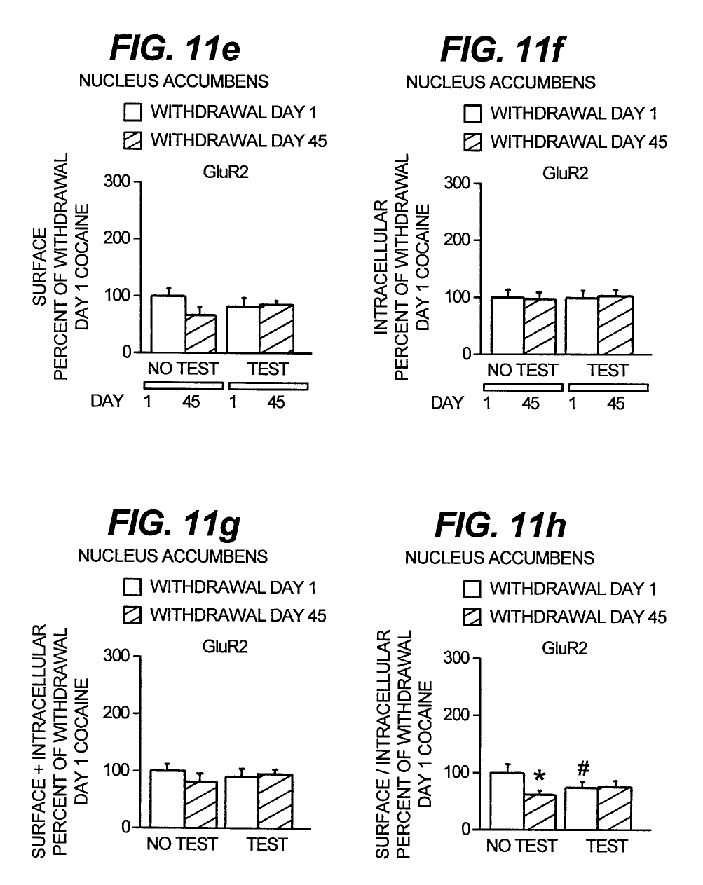

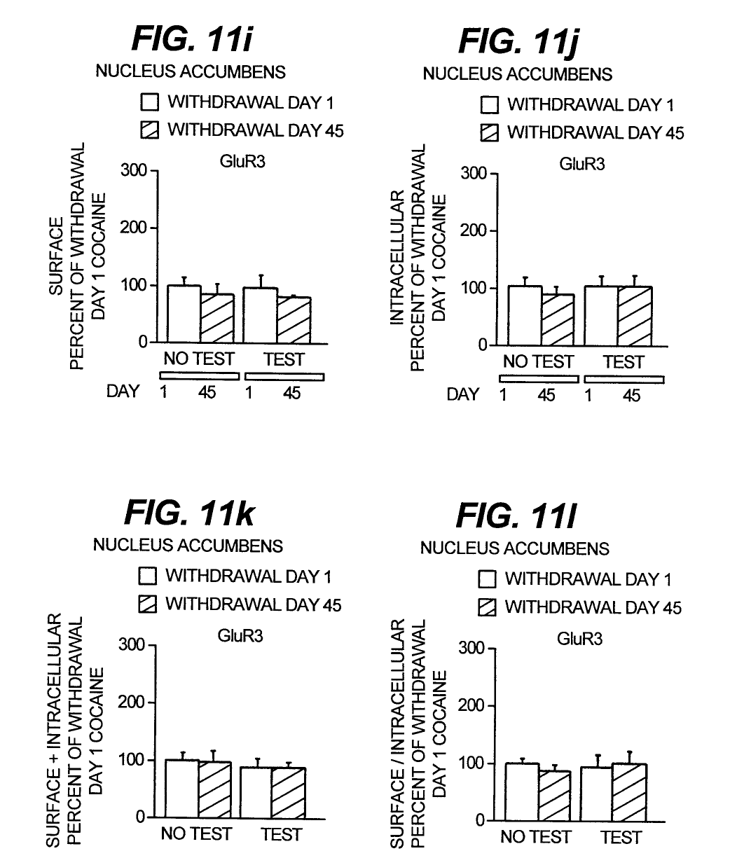

Next, we determined if the time-dependent changes in AMPA receptor expression were influenced by performing a test for cue-induced cocaine-seeking (under extinction conditions). We trained rats to self-administer cocaine as described above. We assessed the brains of 4 groups of rats that were either tested (“test”) or not tested (“no-test”) for cue-induced cocaine-seeking after 1 or 45 days of withdrawal from cocaine; rats in the test condition were killed immediately after the 30-min cocaine-seeking test. We found increased surface and total GluR1 levels on withdrawal day 45 (FIG. 11a-c) in both the test and no-test conditions, replicating results from our first experiment (FIG. 2a-c). “No-test” rats also exhibited a small reduction in the GluR2 surface/intracellular ratio on withdrawal day 45 (FIG. 11h). These data suggest that the test for cocaine-seeking had a minimal effect on accumbens AMPA receptor distribution.

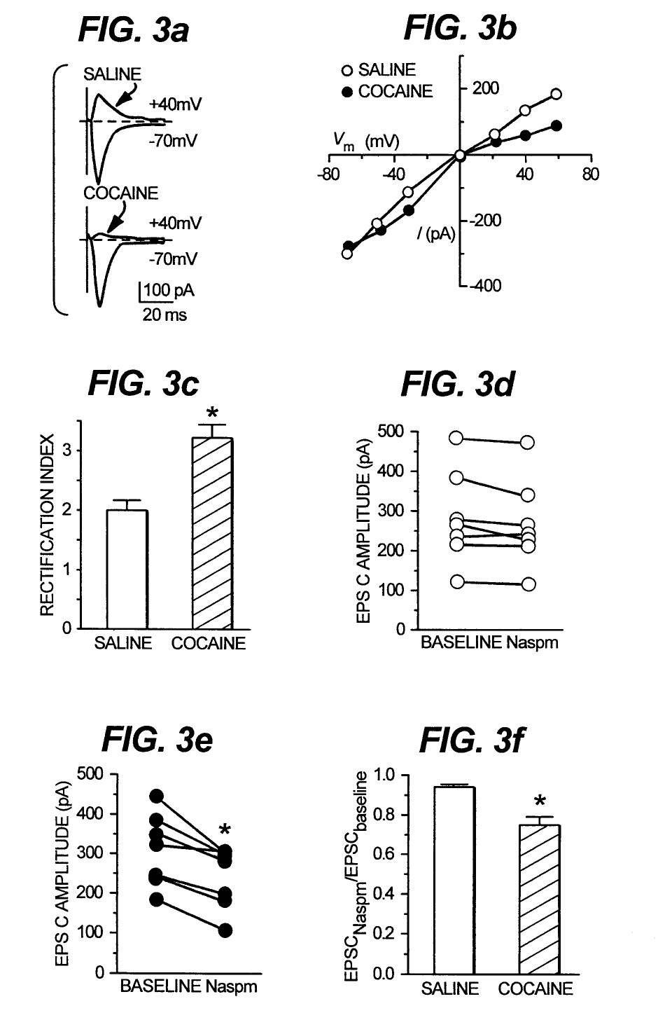

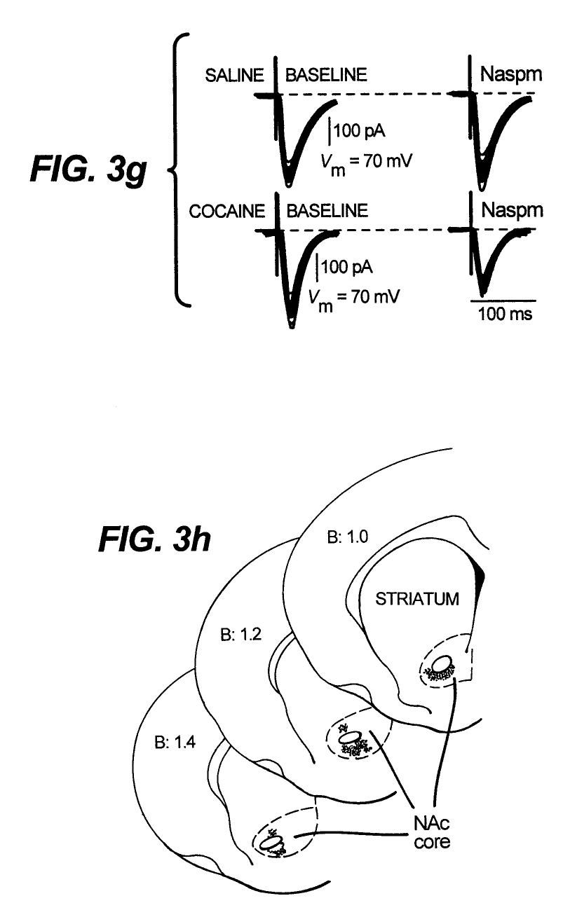

To confirm our biochemical results, we performed whole-cell patch clamp recordings of medium spiny neurons in the accumbens core after 42-47 days of withdrawal from saline or cocaine self-administration. GluR2-lacking AMPA receptors have unique properties: permeability to Ca²⁺ resulting in greater conductance and inwardly rectifying currents due to voltage-dependent block by polyamines^{8,9}. Current-voltage relationships of evoked EPSCs (FIGS. 5-12) in accumbens neurons revealed significantly greater inward rectification in the cocaine-exposed group (FIG. 3, a-c). Furthermore, bath application of 1-naphthylacetylsperimine (Naspm), a selective blocker of GluR2-lacking AMPA receptors, significantly reduced evoked EPSC amplitude only in neurons recorded from the cocaine-exposed group (FIG. 3d-g). Thus, GluR2-lacking AMPA receptors contribute significantly to accumbens synaptic transmission only after prolonged withdrawal from cocaine.

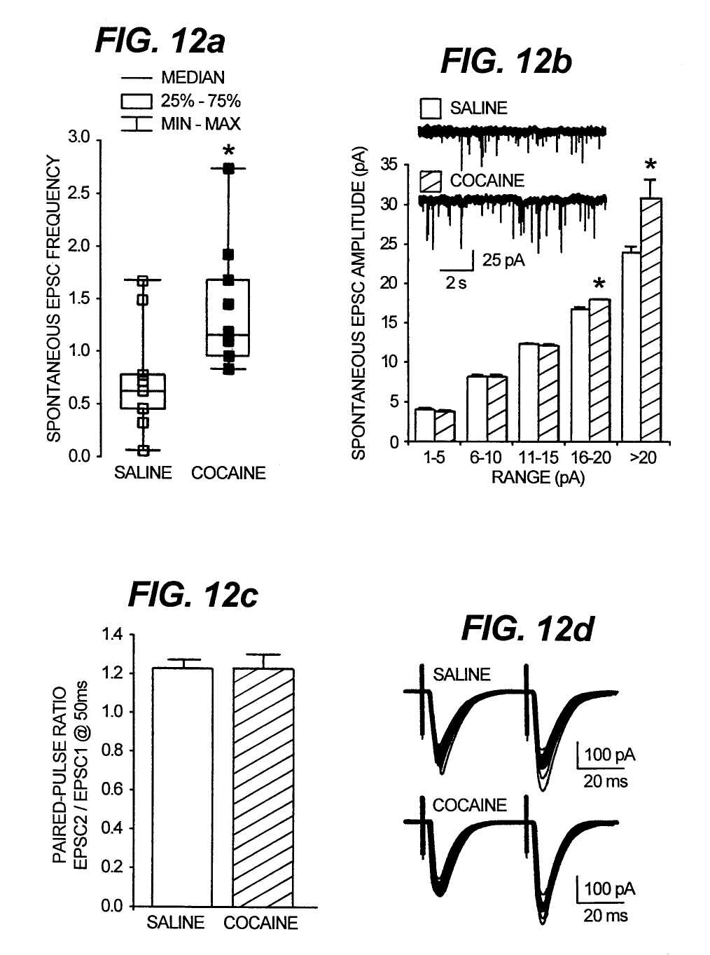

Additionally, we found that neurons from cocaine-exposed rats showed a change in the distribution of spontaneous EPSC (sEPSC) amplitude due to an increased number of high-amplitude sEPSC (Supp. FIG. 8b). Both the results with Naspm and the increased sEPSC amplitude predict enhanced responsiveness of accumbens neurons to excitatory inputs after prolonged withdrawal from cocaine. Neurons from cocaine-exposed rats also showed increased frequency of AMPA receptor-mediated sEPSC (FIG. 12a). This is unlikely to reflect increased release probability, because the paired-pulse ratio did not differ between cocaine- and saline-exposed rats (FIG. 12c,d). Increased sEPSC frequency may be due to the formation of new synaptic contacts in accumbens after withdrawal from cocaine¹⁷.

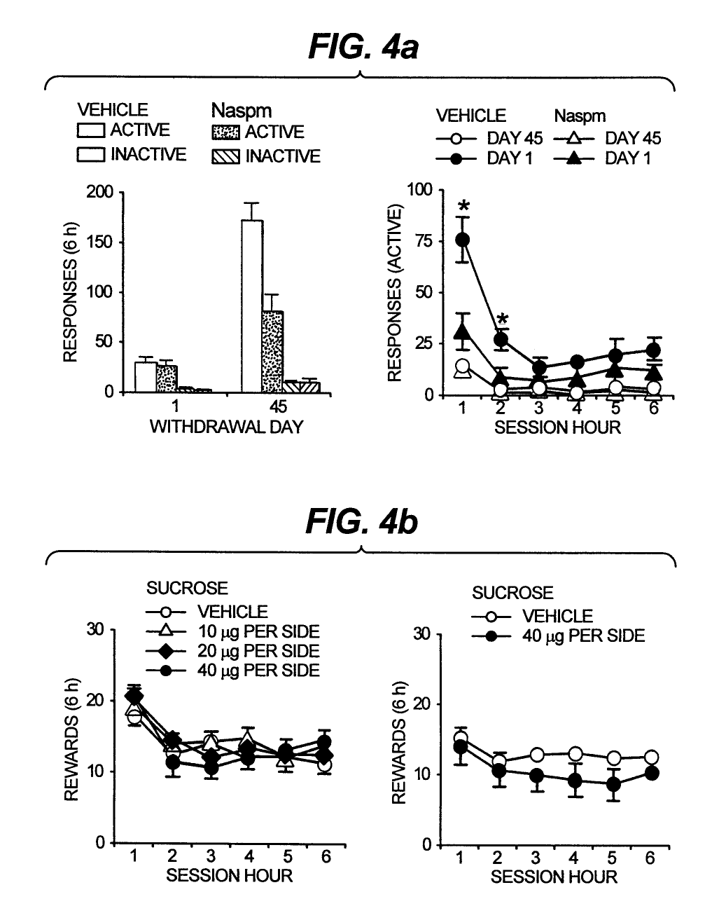

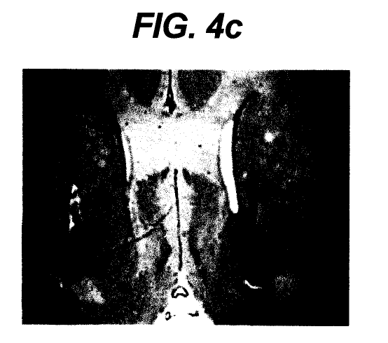

To test the functional role of new GluR2-lacking receptors, we injected Naspm (or vehicle) into the accumbens of cocaine-exposed rats prior to tests for cue-induced cocaine-seeking. Naspm significantly reduced cue-induced cocaine-seeking on withdrawal day 45, demonstrating that GluR2-lacking AMPA receptors mediate the expression of incubation of cocaine craving (FIG. 4a). Naspm did not alter cue-induced cocaine-seeking on withdrawal day 1 (FIG. 4a). This finding is consistent with lack of differences in AMPA receptor subunit expression and distribution on cocaine withdrawal day 1 versus the drug-naïve saline condition (FIG. 2), in which GluR2-lacking receptors are expressed at very low levels and contribute minimally to accumbens synaptic transmission (FIGS. 3 and 6). Naspm did not alter ongoing cocaine or sucrose self-administration (FIG. 4b).

We propose that the synaptic incorporation of GluR2-lacking AMPA receptors enhances responsiveness of accumbens neurons to glutamate inputs from cortical and limbic regions, due to increases in the absolute number of surface AMPA receptors (FIG. 2), as well as the higher conductance of GluR2-lacking AMPA receptors^{8,9}. Thus, when cocaine-associated cues are presented after prolonged withdrawal from cocaine and glutamate is released in the accumbens, accumbens neurons respond more robustly, leading to enhanced cocaine-seeking.

Our results are consistent with a large body of literature implicating increased accumbens AMPA receptor transmission in cocaine-seeking⁵⁻⁷ and psychomotor sensitization^{13,14,18-21} after prolonged withdrawal from cocaine, and with the finding that increased accumbens neuronal activity correlates with the incubation of cocaine craving²². However, our results are different from those of Mead et al.²³ who reported that cue-induced cocaine-seeking after prolonged withdrawal was not decreased in GluR1-knockout mice. These results should be interpreted with caution in light of the potential for compensation during development and/or offsetting changes in other neuronal pathways. Our results also differ from those of Sutton et al.²⁴ who reported that viral over-expression of GluR1 or GluR2 in accumbens shell decreased extinction responding during early withdrawal from cocaine. Many differences exist between our two studies, including focus on core vs. shell, long vs. short withdrawal, and a single vs. multiple extinction tests. An important consideration is that our conclusions are based on measuring and manipulating endogenous surface AMPA receptors.

Recent work has highlighted the importance of GluR2-lacking AMPA receptors in long-term potentiation (LTP) and depression (LTD), experience-dependent plasticity and synaptic scaling, a form of homeostatic plasticity wherein prolonged activity blockade causes enhanced excitatory synaptic transmission^{8,9}. Synaptic scaling may have parallels to our model. After withdrawal from cocaine, cortical areas providing excitatory input to the accumbens show metabolic hypoactivity^{10,25}, raising the possibility that accumbens GluR2-lacking AMPA receptors scale up as a homeostatic response to prolonged decreases in synaptic activation. Scaling-induced increases in GluR1 have been reported to occur through increased dendritic GluR1 synthesis as well as decreased GluR1 protein stability²⁷.

In conclusion, we demonstrated that GluR2-lacking AMPA receptors are produced in the accumbens during prolonged abstinence from cocaine and play a causal role in the incubation of cocaine craving. Our work adds to a growing consensus that perturbations in synaptic transmission during disease states cause compensatory changes in AMPA receptor subunit composition that alter the properties of neuronal networks^{8,9}. For cocaine addiction, production of GluR2-lacking AMPA receptors may exacerbate disease processes by increasing the reactivity of accumbens neurons to cocaine-associated cues that promote craving and relapse. A question for future research is whether accumbens GluR2-lacking receptors also contribute to drug- and stress-induced cocaine craving and relapse that also occur after prolonged abstinence^{3,10}. Finally, our results, and those of Lüischer and colleagues^{28,29} on the formation of GluR2-lacking AMPA receptors in VTA after acute cocaine exposure, suggest that these receptors represent a new drug target for addiction treatment.

### Methods Summary

All procedures are based on our previous work^{2,3,13,14,30} and are described in detail with respect to FIGS. 5-12.

### Behavioral Procedures:

Male rats were trained to nose-poke (biochemical and electrophysiology experiments) or lever-press (Naspm accumbens injections experiment) for 6-h/day for 10-12 days; each cocaine infusion was paired with a tone-light or light cue. After self-administration training, the rats were tested for cue-induced cocaine-seeking after 1 or 45 withdrawal days. During testing, lever or nose-poke responding led to contingent presentations of the cue previously paired with cocaine infusions, but not cocaine. Responding on the previously active lever or hole was the operational measure of cocaine-seeking.

### Biochemistry:

After the appropriate withdrawal period (or immediately after the drug-seeking test in FIG. 11), the rats were decapitated. The accumbens was rapidly dissected and brain slices (400 μM) prepared with a tissue chopper. Crosslinking of slices with BS³ (30 min), subsequent tissue processing, and quantification of surface and intracellular protein levels by SDS-PAGE and Western blotting were performed as described below with respect to FIGS. 5-12. Values for surface, intracellular and total receptor subunit levels were normalized to total protein in the lane determined using Ponceau S.

### Electrophysiology:

Coronal slices (300 μm thick) containing the accumbens were obtained after 42-47 days of withdrawal from saline or cocaine self-administration. Recordings were conducted in voltage clamp configuration at 33-35° C. with patch electrodes filled with Cs-gluconate, spermine (0.1 mM) and QX-314 (1 mM). Medium spiny neuron synaptic responses were elicited by local stimulation of excitatory inputs using a bipolar electrode. Stimulation intensity (0.05 to 0.3 mA) was based on the minimum amount of current necessary to elicit a synaptic response with <15% variability in amplitude 10 min after obtaining the whole-cell configuration. Both spontaneous and evoked EPSCs were collected before and after 10 min bath application of Naspm (100-200 μM).

As shown in FIG. 5, cocaine-exposed rats at intermediate withdrawal times (3 and 21 days) show intermediate changes in GluR1 expression and distribution. Rats were trained to self-administer saline or cocaine and killed after 3 or 21 days of withdrawal. Saline-exposed rats did not differ at the two withdrawal times and were therefore pooled. Protein crosslinking analysis was performed using a dissection that included the entire accumbens. To enable comparison of AMPA receptor parameters on withdrawal days 3 and 21 with results from withdrawal days 1 and 45 (FIG. 2), mean values for cocaine-exposed rats on withdrawal days 1 and 45 (expressed as percent of saline rats on withdrawal day 1) are shown by dashed and solid lines, respectively, in each graph.

The one-way ANOVA results reported below are based on the pooled saline group, cocaine withdrawal day 3, and cocaine withdrawal day 21; significant results were followed by post-hoc tests. GluR1: Cell surface (a) and total (c) GluR1 levels were increased in cocaine-exposed rats on withdrawal day 21 compared to withdrawal day 3 and saline-exposed group (F_{2,26}=22.4, and F_{2,26}=3.6, respectively, p values<0.05). Note that day 3 and day 21 values for these parameters were higher than day 1 values (dotted lines) and lower than day 45 values (solid lines). These results, together with FIG. 2, indicate that GluR1 levels increase gradually over 45 days of withdrawal, but much of the increase occurs between withdrawal days 21 and 45.

There were no group differences in intracellular GluR1 (b) or the GluR1 surface/intracellular ratio (d). GluR2: There were no group differences in cell surface (e), intracellular (f), or total (g) GluR2 levels. The GluR2 surface/intracellular ratio (h) was greater in cocaine-exposed rats on withdrawal day 3 compared with pooled saline-exposed rats (F_{2,26}=7.4, p<0.05). Together with FIG. 2, these results indicate that there are no substantial changes in GluR2 expression or distribution over 45 days of withdrawal. GluR3: The GluR3 surface/intracellular ratio (l) was increased in cocaine-exposed rats on withdrawal days 3 and 21 compared with saline-exposed rats (F_{2,26}=4.0, p<0.05). Similarly, the GluR3 surface/intracellular ratio was increased in cocaine-exposed rats on withdrawal day 45 compared with the saline-exposed group and there was a trend towards an increase on withdrawal day 1 (FIG. 2).

Values on days 3 and 21 were within the same range as values for days 1 and 45 (dotted and sold lines in panel l, respectively). Together, these results indicate that the GluR3 surface/intracellular ratio is increased after withdrawal from cocaine in a time-independent manner. Surface GluR3 (i) did not increase significantly on days 3 and 21 compared with saline-exposed group, whereas time-independent increases in this parameter were observed in the cocaine-exposed group on withdrawal days 1 and 45 (dotted and sold lines in panel i and FIG. 2). Intracellular (j) and total (k) GluR3 did not change significantly on withdrawal days 3 and 21, consistent no changes on withdrawal days 1 and 45 (FIG. 2). Surface, intracellular and total GluR1-3 values were normalized to total protein in the lane determined using Ponceau S. The surface/intracellular ratio is independent of total protein loaded on the gel. Data (mean±S.E.M, n=5-15 per group) are expressed as a percentage of the pooled saline group. * Different from the other conditions, p<0.05; # Different from pooled saline, p<0.05.

FIG. 6 shows quantitative co-immunoprecipitation of AMPA receptor subunits in the nucleus accumbens after prolonged withdrawal from cocaine self-administration. AMPA receptor subunit composition was compared in accumbens tissue from (a) saline- and (b) cocaine-exposed rats that were killed on withdrawal day 45, using the quantitative co-immunoprecipitation method described in the Extended Methods herein. Immunoblots (IB) show the percentage of AMPA receptor subunits remaining (unbound fraction) after immunoprecipitation (IP) of solubilized accumbens tissue. The AMPA receptor subunit antibodies used to IP are indicated at the top of a and b panels. The antibodies used to IB are indicated on the left of a and b panels. The left two lanes in each row show immunoblotting of IgG control IP'ed tissue and indicate the range of immunoreactivity detected. The percent remaining is calculated from the standard curve generated by controls of 5% (shown), 25%, 50%, 75% and 100% (shown) run on each blot.

In saline-exposed rats, unbound GluR1 was below the limit of detection (not detectable; N.D.) after IP with either GluR2 or GluR2/3 antibodies, indicating that nearly all GluR1 is associated with GluR2. In contrast, three sets of results indicated decreased association between GluR2 and GluR1 after prolonged withdrawal from cocaine self-administration: 1) After GluR1 IP, cocaine-exposed rats show an increase in GluR2 and GluR2/3 remaining in the unbound fraction (53 and 43% in control rats and 70 and 55% in cocaine rats, respectively), indicating an increase in GluR2 and GluR3 not associated with GluR1. 2) After GluR2 IP, cocaine-exposed rats show an increase in GluR1 remaining in the unbound fraction (N.D. in controls and 8% in cocaine rats), indicating an increase in GluR1 not associated with GluR2 (GluR1/3 or homomeric GluR1; GluR4 is not present in medium spiny neurons—see Extended Methods for references). 3) After GluR2/3+4 IP, cocaine-exposed rats show an increase in GluR1 in the unbound fraction (N.D. in controls and 6% in cocaine rats), indicating an increase in GluR1 not associated with any other subunit (homomeric GluR1).

These data do not permit conclusions about the magnitude of the increase in GluR2-lacking AMPA receptors, because the absolute amount of GluR1 protein increased after 45 days of withdrawal from cocaine (FIG. 2). One result was inconsistent with our other findings. After IP for GluR2/3, cocaine-exposed rats did not show an increase in unbound GluR1 (7% for controls, 6% for cocaine). Also notable is a decrease in unbound GluR3 in cocaine-exposed rats after IP for GluR2 (14% for controls, 6% for cocaine), suggesting increased association between GluR2 and GluR3 in cocaine rats on withdrawal day 45. This may seem inconsistent with data in FIG. 2 indicating no increase in GluR2 expression after prolonged withdrawal from cocaine. It is possible, however, that the same overall level of GluR2 expression is maintained by substituting some GluR2/3 receptors for GluR1/2 receptors. This would explain the IP results, as well as the lack of change in GluR2 expression and the increase in GluR3 surface expression (although some new GluR3 on the surface is likely GluR1/3) in FIG. 2.

As shown in FIG. 7, the effect of cocaine self-administration and subsequent withdrawal on AMPA receptor subunit expression and distribution in the ventral tegmental area (VTA). Saline- and cocaine-exposed rats were compared on withdrawal days 1 and 45. No significant drug exposure by withdrawal day interactions were observed for GluR1 (a-d) or GluR2 (e-h). Cell surface (i), intracellular (j), and total (k) GluR3 levels were increased 45 days after withdrawal from cocaine (F_{1,31}=4.9, F_{1,31}=4.9, and F_{1,31}=5.0, respectively, p values<0.05 for drug exposure by withdrawal day interaction) but the surface/intracellular ratio (l) was not changed. Data (mean±S.E.M) are expressed as a percentage of the saline-exposed group on withdrawal day 1; n=6-9 per group. Surface, intracellular and total GluR1-3 values were normalized to total protein in the lane determined using Ponceau S. The VTA was dissected from a 2 mm slice obtained with a brain matrix (approximately −4.50 to −6.50 mm from Bregma). * Different from the other groups, p<0.05.

As shown in FIG. 8, cocaine self-administration and subsequent prolonged withdrawal does not significantly alter AMPA receptor subunit expression and distribution in the cingulate cortex. Saline- and cocaine-exposed rats were compared on withdrawal day 45. There were no significant differences with respect to surface (S), intracellular (I), and total (S+I) protein levels or the surface/intracellular ratio (S/I) for GluR1 (a), GluR2 (b), or GluR3 (c). Data (mean±S.E.M) are expressed as a percentage of saline-exposed groups on withdrawal day 45; n=7 per group. Surface, intracellular and total GluR1-3 values were normalized to total protein in the lane determined using Ponceau S. The cingulate cortex was dissected from a 2 mm slice obtained with a brain matrix (approximately 2.5 to 4.5 mm from Bregma) by harvesting cortical tissue dorsal to the prelimbic cortex. (d) Representative Western blots for GluR1, GluR2, and GluR3.

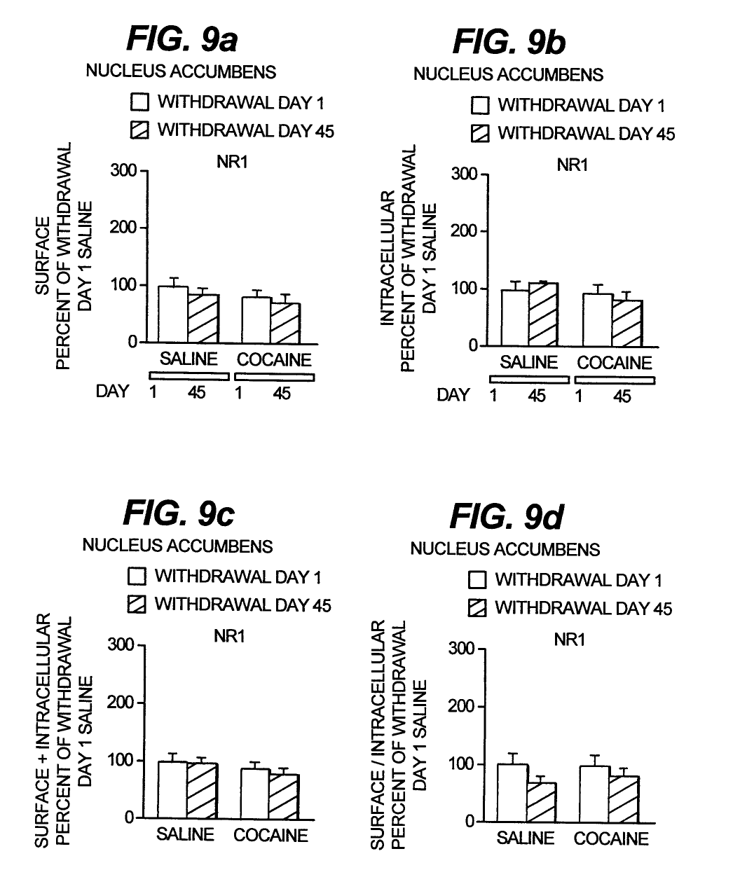

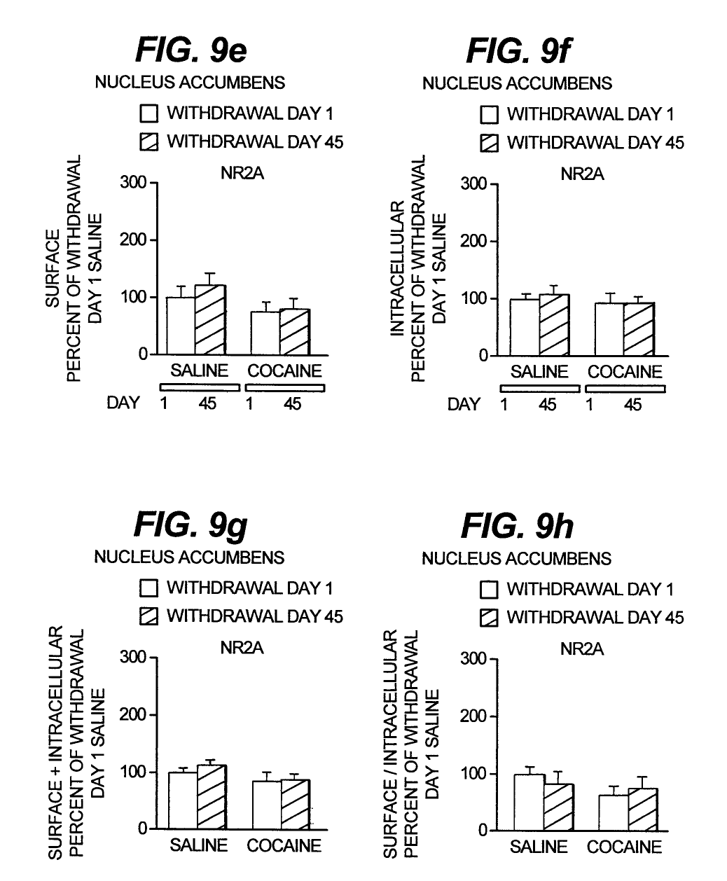

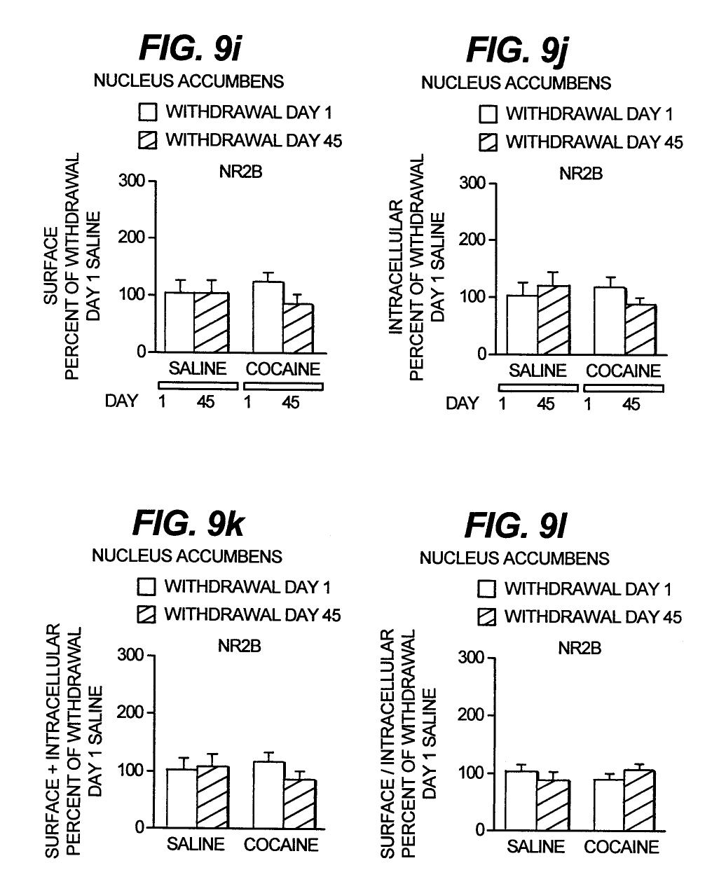

As shown in FIG. 9, cocaine self-administration and subsequent prolonged withdrawal does not significantly alter NMDA receptor subunit expression and distribution in the nucleus accumbens. Saline- and cocaine-exposed rats were compared on withdrawal days 1 and 45. There were no significant differences with respect to surface, intracellular and total (surface+intracellular) protein levels or surface/intracellular ratios for NR1 (a-d), NR2A (e-h), or NR2B (i-l). Data (mean±S.E.M) are expressed as a percentage of saline-exposed group on withdrawal day 1; n=7-11 per group. Surface, intracellular and total values were normalized to total protein in the lane determined using Ponceau S.

As shown in FIG. 10, AMPA receptor adaptations after withdrawal from cocaine self-administration occur primarily in the nucleus accumbens core subregion. Cocaine-exposed rats were compared on withdrawal days 1 and 45. AMPA receptor adaptations in the core paralleled those observed when the entire accumbens was dissected (data for entire accumbens are shown in FIG. 2). (a) In core, surface (S), intracellular (I), and total (S+I) GluR1 levels were increased on withdrawal day 45 compared to withdrawal day 1 (F_{1,14}=36.5, F_{1,14}=12.5, and F_{1,14}=72.6, p<0.01, respectively, p values<0.01). (b) In core, the GluR2 surface/intracellular ratio (S/I) was decreased after 45 days of withdrawal from cocaine (F_{1,14}=8.2, p<0.05). (c) In core, GluR3 intracellular and total levels were increased after 45 days of withdrawal from cocaine (F_{1,14}=8.1 and F_{1,16}=5.9, respectively, p values<0.05). (d) In shell, GluR1 surface levels were increased after 45 days of withdrawal from cocaine (F_{1,13}=3.3, p<0.05). (e, f) In shell, there were no significant changes in expression or distribution of GluR2 or GluR3. GluR3 surface levels in the entire accumbens were increased in cocaine-exposed rats on withdrawal days 1 and 45 compared with the saline-exposed group (FIG. 2) but this effect cannot be evaluated in the present data set, which strictly compares cocaine-exposed rats on withdrawal days 1 and 45. Data (mean±S.E.M) are expressed as a percentage of cocaine-exposed rats on withdrawal day 1; n=7-9 per group. Surface, intracellular and total GluR1-3 values were normalized to total protein in the lane determined using Ponceau S. * Different from withdrawal day 1, p<0.05.

As shown in FIG. 11, a 30 min cue-induced cocaine-seeking test has little effect on AMPA receptor expression and redistribution in the nucleus accumbens after withdrawal from cocaine self-administration. All rats shown were trained to self-administer cocaine. Surface (a), intracellular (b), and total (c) levels of GluR1 were increased on withdrawal day 45 regardless of whether rats received an extinction test for cue-induced cocaine seeking on withdrawal day 1 or 45 (test) or were killed on these days without a test (no test) (main effects of withdrawal day: F_{1,25}=9.9, F_{1,25}=11.0 and F_{1,25}=10.7, respectively, p values<0.01). Surface (e), intracellular (f), and total (g) levels of GluR2 did not differ between test and no test groups. The GluR2 surface/intracellular ratio (h) changed as indicated by a significant test condition (test, no test) by withdrawal day interaction (F_{1,25}=4.6, p<0.05). The GluR2 surface/intracellular ratio was lower on withdrawal day 45 than on withdrawal day 1 in the no test group (*p<0.05) and lower on withdrawal day 1 in the test group compared to withdrawal day 1 in the no test group (^{#}p<0.05). (i-l) No significant differences were observed for GluR3 between test and no test groups. Data (mean±S.E.M) are expressed as a percentage of cocaine-exposed rats on withdrawal day 1 in the no test group; n=7-8 per group. Surface, intracellular, and total GluR1-3 levels were normalized to total protein in the lane determined using Ponceau S.

As shown in FIG. 12, Nucleus accumbens medium spiny neurons recorded after prolonged withdrawal from cocaine self-administration exhibit increased frequency and amplitude of spontaneous EPSCs (sEPSC) compared with the saline-exposed group but no change in the paired-pulse facilitation ratio. Recordings were performed from medium spiny neurons of the nucleus accumbens after 42-47 days of withdrawal from saline or cocaine self-administration. FIG. 12a shows medium spiny neurons recorded from cocaine-exposed rats (n=11 from 4 rats) exhibit a significant increase in the frequency of AMPA receptor-mediated sEPSCs when compared with the saline-exposed group (n=9 from 3 rats) (expressed as number of events/s, box-plot, t₁₈₌=2.51, *p<0.05).

FIG. 12b is a bar graph summarizing the mean amplitude by range of sEPSC size obtained from the same recordings used to compute the frequency data shown in FIG. 12a. Medium spiny neurons recorded from saline- and cocaine-exposed rats showed similar sEPSC amplitude at sEPSC size ranges <15 pA. However, amplitude analysis of sEPSC size >15 pA revealed a significant increase in the cocaine group compared with the saline group (t₁₈=2.25, p<0.05 at 15-20 pA, and t₁₈=2.34, p<0.05 at >20 pA).

The insets of FIG. 12 show two traces of sEPSCs recorded from nucleus accumbens neurons after 45 days of withdrawal from saline or cocaine self-administration. FIG. 12c is a bar graph summarizing the presence of paired-pulse facilitation in both groups of neurons. The paired-pulse facilitation ratio was calculated as the ratio of the amplitude of evoked EPSC₂/EPSC₁. Accumbens medium spiny neurons recorded from rats in the cocaine-exposed (n=11) and saline-exposed (n=9) groups showed similar paired-pulse facilitation ratios of ˜1.2 (i.e., amplitude of EPSC₂ is 20% larger than EPSC₁). FIG. 12d shows traces of evoked EPSCs recorded from cocaine- and saline-exposed rats showing the presence of paired-pulse facilitation. * Different from saline, p<0.05.

### Extended Methods

### Subjects

The subjects were male Sprague Dawley rats (Harlan, Indianapolis, Ind.; biochemical experiments) and Long Evans rats (Charles River, Raleigh, N.C.; Naspm experiment performed at the IRP/NIDA) weighing 250-275 g upon arrival. The rats were housed individually on a reverse 12 h/12 h light-dark cycle (lights out at 0900 hours). Rats had access to water and food ad libitum at all times unless specified. All procedures followed the “Principles of Laboratory Animal Care” (NIH publication no. 86-23, 1996) and were approved by the local Animal Care and Use Committees. The cocaine- and saline-trained rats were implanted with either intravenous catheters or intravenous catheters plus bilateral cannulae aimed at the nucleus accumbens (see below). The inclusion of saline-exposed control rats that are drug-free but still exposed to the same experimental conditions was used to control for effects of ageing on our molecular measures.

### Surgical Procedures

The rats were anesthetized using isoflurane gas (Henry Schein, Melville, N.Y.) and flunixin meglumine was administered before surgery (2 mg/kg, i.p.) as an analgesic. A silastic catheter was inserted into the right auricle through the external jugular vein, passed under the skin and fixed in the mid-scapular region. The rats recovered from surgery for at least seven days prior to beginning self-administration training sessions. During this time, catheters were flushed every 24-48 h with sterile 0.9% saline. The rats undergoing intravenous self-administration experiments together with intracranial infusions (Naspm accumbens injection experiment) were anesthetized with sodium pentobarbital and chloral hydrate (60 and 25 mg/kg, i.p.), and permanent guide cannulae (23-gauge, Plastics One, Roanoke, Va.) were implanted bilaterally 1 mm above the nucleus accumbens and were aimed at the core sub-region (coordinates: 6° angle aimed medially, AP +1.7, ML ±2.5, and DV −6.0)³¹.

Following cannulae implantation, silastic catheters were inserted into the jugular vein, attached to a modified 22-gauge cannula and mounted to the rat's skull with dental cement (see ref.^{32,33}) Buprenorphine (0.1 mg/kg, s.c.) was given after surgery as an analgesic and the rats recovered for 7-10 days before behavioral testing began. During the recovery and training phases for these rats, catheters were flushed every 24-48 h with sterile 0.9% saline+the antibiotic Gentamicin (0.08 mg/mL).

### Intracranial Injections

The intracranial injection methods were based on our previous studies^{32,33}. 1-Naphthylacetyl spermine trihydrochloride (Naspm; Sigma-Aldrich, St. Louis, Mo.) was dissolved in phosphate buffered saline (PBS). Injections of vehicle or Naspm (10, 20 and 40 μg/side) were made with Hamilton syringes (Hamilton, Reno, Nev.) that were connected to 30-gauge injectors (Plastics One, Roanoke, Va.). A volume of 0.5 μL was infused into each side over 1 min and the injector was left in place for 1 min after the injections. The rats were tested within 15 min after intracranial injections. The Naspm doses were based on previous reports^{34,35} and on an initial study with sucrose-trained rats (see below). At the end of the experiments, the rats were deeply anaesthetized, their brains were removed, and coronal sections (40 μm) were sliced on a cryostat and stained with Cresyl Violet (ICN Biomedicals Inc., Aurora, Ohio). Cannulae placements were verified under a microscope and their anatomical location is depicted in FIG. 4c.

### Cocaine Self-Administration Training

Following recovery from surgery, the rats were allowed to self-administer for 6 h/day cocaine or saline for 10 days (biochemical and electrophysiological experiments) or for 10-12 days (Naspm accumbens injections experiment). The self-administration chambers (MED Associates, St. Albans, Vt.) were located in sound-attenuating cabinets. Rats were either housed chronically in these chambers (for the Naspm experiments), or they were placed daily in these chambers; sessions began approximately at the start of the dark cycle. For the Naspm experiments, the self-administration chambers were equipped with two levers. Presses on one (active, retractable) activated the infusion pump and delivered an infusion of cocaine (0.75 mg/kg); presses on the other (inactive, stationary) had no effects. A fixed-ratio-1 reinforcement schedule was used, with a 40-s timeout period after each infusion; cocaine infusions were accompanied by a 5-s tone-light cue.

Each session began with the insertion of the active lever and the illumination of a houselight that remained on for the entire session. At the end of each session, the houselight was turned off and the active lever retracted. To facilitate the acquisition of cocaine self-administration, food was removed from the chambers during the 6-h sessions of the first 3 training days. The number of cocaine infusions was limited to 20/h to prevent overdose. For all other experiments, the self-administration chambers were equipped with 2 holes located 2 cm above the floor. Nose-poking in the active hole activated the infusion pump and delivered an infusion of saline or cocaine (0.5 mg/kg); nose-poking in the inactive hole had no consequences. In addition to activating the infusion pump, nose-poking in the active hole was paired with a 5-s discrete light cue, located inside the nose hole.

A time-out period of 10 s was used during the first hour or for the first 10 infusions (whichever occurred first) and then the time-out period was extended to 30 s for the remaining hours, to prevent cocaine overdose. Food and water were present at all times. For sucrose self-administration (results shown in FIG. 4b, n=10), procedures were the same as those described above for cocaine self-administration for the Naspm experiment, except that active lever presses led to the delivery of 0.75 mL of 10% sucrose solution into receptacles located near the lever. After stable sucrose self-administration behavior was achieved, the rats were injected every other day with vehicle or Naspm (10, 20 or 40 μg/side) into the accumbens 15 min before the test sessions, which were separated by regular training days. The order of the injections of the vehicle and the different Naspm doses was counterbalanced. Naspm (40 μg/side) or its vehicle was also injected during cocaine self-administration in a sub-group of rats (n=5), as described above for sucrose.

### Tests for Cue-Induced Cocaine Seeking

At the end of the training phase, the rats were returned to the animal facility where they remained for 1 or 45 days (the rats in the late withdrawal period were handled several times per week). After this time, they were brought back to the self-administration chambers, where they were tested for cue-induced cocaine-seeking under extinction conditions; that is, all conditions were the same as during training, with the exception that responding on the active device was not reinforced with drug. During the extinction tests, lever or nose-poke responding led to contingent presentations of the tone-light or light cue previously paired with cocaine infusions. The number of responses in the previously active lever or hole was used as a measure of cocaine-seeking.

### Protein Crosslinking

Each experimental group consisted of 7-18 rats, with the exception of cocaine withdrawal day 21 (n=5). The rats were decapitated, their brains were rapidly removed, and the nucleus accumbens (or other region of interest) was dissected on ice from a 2 mm coronal section obtained using a brain matrix. Tissue was immediately chopped into 400 μm slices using a McIllwain tissue chopper (Vibratome, St. Louis, Mo.). Slices were added to Eppendorf tubes containing ice-cold artificial CSF which was spiked with 2 mM bis(sulfosuccinimidyl)suberate (BS³; Pierce Biotechnology, Rockford, Ill.) immediately after addition of the tissue. Slices were crosslinked for 30 min at 4° C. with gentle agitation. Crosslinking was terminated by addition of 100 mM glycine (10 min at 4° C.). Slices were pelleted by brief centrifugation, re-suspended in ice-cold lysis buffer containing protease and phosphatase inhibitors, sonciated for 5 sec to disrupt tissue, and centrifuged (20,800×g, 2 min) as described previously^{36,37}.

The supernatant fraction was aliquoted and stored at −80° C. BS³ is a membrane impermeant crosslinking agent. Therefore, it selectively crosslinks cell surface proteins, forming high molecular weight aggregates. Intracellular proteins are not modified and thus retain their normal molecular weight. This enables surface and intracellular pools of a particular protein to be distinguished by SDS-PAGE and Western blotting. Variants of this assay have been used previously to measure glutamate receptor surface expression in dissociated cells and brain slices³⁸⁻⁴⁶. We adapted the assay to detect receptor redistribution produced after in vivo treatments³⁶. We and others have shown that incubation of brain slices or dissociated cultures with BS³ does not crosslink intracellular proteins (e.g., actin, tubulin, synapsin, tyrosine hydroxylase, and protein kinases) unless BS³ crosslinking is performed in a lysed preparation^{36-42,44,45}.

### Western Blot Analysis of Glutamate Receptor Subunits in Crosslinked Tissue

Samples were run on 4-15% gradient Tris-HCl gels (Bio-Rad, Hercules, Calif.) under reducing conditions, proteins were transferred to PVDF membranes, and membranes were washed in Tris buffered saline (TBS) and blocked with 1% goat serum/5% nonfat dry milk in TBS-Tween-20 (TBS-T). Membranes were incubated overnight at 4° C. with the following 1° antibodies: GluR1 (1:500; Millipore, Billerica, Mass.), GluR2 (1:1000, Millipore), GluR3 (1:500; Millipore), NR1 (1:500; Millipore), NR2A (1:2000, Santa Cruz Biotechnology, Santa Cruz, Calif.), and NR2B (1:1000; Millipore).

Not all lots of the NR1 antibody gave satisfactory results in this assay. Membranes were washed with TBS-T solution, incubated for 60 min with HRP-conjugated anti-rabbit IgG or anti-mouse IgG (1:10,000; Upstate Biotechnology, Lake Placid, N.Y.), washed with TBS-T, rinsed with ddH₂O, and immersed in chemiluminescence (ECL) detecting substrate (Amersham GE, Piscataway, N.J.). Images were captured with Versa Doc Imaging Software (Bio-Rad). Diffuse densities of surface and intracellular bands were determined with Quantity One software (Bio-Rad). Values for surface, intracellular and total (surface+intracellular) protein levels were normalized to total lane protein determined using Ponceau S (Sigma-Aldrich) and analyzed with TotalLab (Nonlinear Dynamics, Newcastle, UK). The surface/intracellular ratio did not require normalization, because both values are determined in the same lane.

### Quantitative Co-Immunoprecipitation

Using the methods developed by Wenthold and colleagues^{47,48} and with the help of advice from the Wenthold laboratory, we quantitatively determined AMPA receptor subunit composition in the accumbens after 45 days of withdrawal from cocaine or saline self-administration. Briefly, the rats were decapitated, their brains were rapidly removed, and the accumbens was dissected on ice from a 2 mm coronal section obtained using a brain matrix. Tissue from 3 rats from each experimental group was combined and homogenized in 50 mM Tris-HCl pH 7.4 containing protease inhibitor cocktail (Calbiochem, San Diego, Calif.) (40-60 mg wet weight/mL). The membranes were sedimented by centrifugation at 100,000×g for 30 min at 4° C. The pellet was then solubilized with 1% Triton X-100 in 50 mM Tris-HCl pH 7.4 containing 1 mM EDTA for 45 min at 37° C. Insoluble material was removed by centrifugation at 100,000×g for 30 min at 4° C. The supernatant was stored at −80° C. until use.

For co-immunoprecipitation, 3-5 μg of antibody (GluR1, GluR2, GluR2/3, or GluR4) or an equal amount of control IgG was incubated with 10-20 μL of 50% protein A agarose slurry (Pierce, Rockford, Ill.) for 4 h at 4° C. The pellet was collected by centrifugation at 1000×g for 30 s and washed 3 times with TBS 0.1% Triton X-100. 100 μL of membrane prep was incubated with the washed pellet overnight at 4° C. The agarose bound antibody was pelleted by centrifugation at 1000×g for 30 sec. This created two fractions, the bound (pellet) and unbound (supernatant). The unbound fraction was then subjected to another round of immunoprecipitation.

Two rounds of immunoprecipitation pulled down >95% of the target AMPA receptor subunit (e.g., in FIG. 6, after IP for GluR1, no GluR1 is detected in the unbound fraction by immunoblotting). After the final immunoprecipitation, the unbound fraction was mixed with an equal volume of sample treatment buffer (Invitrogen, Carlsbad, Calif.) and heated to 70° C. for 10 min. For Western analysis, samples were run on 4-12% Bis-Tris gels (Invitrogen) and transferred to PVDF membranes for immunoblotting. Membranes were washed in dH₂O and blocked with 1% goat serum with 5% Carnation milk in 0.05% Tween-20 in TBS, pH 7.4 for 1 h at room temperature. Membranes were then incubated with subunit-specific antibodies (Millipore: GluR1, 1:500; GluR2/3, 1:2000; GluR2, 1:1000; GluR3, 1:500) overnight at 4° C. We did not immunoprobe for GluR4 because it is not present in medium spiny neurons^{49,50}; consistent with this, after IP for GluR4, 100% of GluR1, GluR2 and GluR3 is detected in the unbound fraction (FIG. 6).

Membranes were then washed with TBS-Tween solution, incubated for 60 min with HRP-conjugated anti-rabbit IgG or anti-mouse IgG (1:10,000; Upstate Biotechnology, Lake Placid, N.Y.), and washed again with TBS-Tween, followed by TBS. Membranes were then rinsed with dH₂O, immersed in chemiluminescence (ECL) detecting substrate (Amersham GE) for 1 min, and visualized with VersaDoc imaging software (Bio-Rad) (between 5 and 60 s, depending on the antibody). Diffuse densities of bands were determined using Quantity One software (Bio-Rad). The percent of total AMPA receptor subunit remaining in the unbound fraction was calculated based on the standard curve created from control IgG immunoprecipitated tissue, as described herein with respect to FIG. 6.

### Electrophysiology

As previously reported⁵¹ the rats were anesthetized with chloral hydrate (400 mg/kg, i.p.) before being decapitated. Brains were rapidly removed into ice-cold artificial cerebral spinal fluid (aCSF) containing (in mM): 125 NaCl, 25 NaHCO₃, 12.5 glucose, 3.5 KCl, 1.25 NaH₂PO₄, 0.5 CaCl₂, 3 MgCl₂, 0.05 APV, and 0.05 picrotoxin (pH 7.45, 295-305 mOsm). Coronal slices (300 μm thick) containing the nucleus accumbens were cut in ice-cold aCSF with a Vibratome, and incubated in warm (˜35° C.) aCSF solution constantly oxygenated with 95% O₂-5% CO₂ for at least 60 min before recording. In the recording aCSF (delivered at 2 ml/min), CaCl₂ was increased to 2 mM and MgCl₂ was decreased to 1 mM. Patch pipettes (6-9 MΩ) were pulled from 1.5 mm borosilicate glass capillaries (WPI, Sarasota, Fla.) with a horizontal puller (Model P97, Sutter Instrument, Novato, Calif.), and filled with a solution containing 0.125% Neurobiotin and (in mM): 140 Cs-gluconate, 10 HEPES, 2 MgCl₂, 3 Na₂-ATP, 0.3 GTP, 0.1 spermine, 1 QX-314 (pH 7.3, 280-285 mOsm). All chemicals and drugs were purchased from Sigma-Aldrich.

Nucleus accumbens medium spiny neurons from the core region were identified under visual guidance using infrared-differential interference contrast (IR-DIC) video microscopy with a 40× water-immersion objective (Olympus BX51-WI). The image was detected with an IR-sensitive CCD camera and displayed on a monitor. Whole-cell patch-clamp recordings were performed with a computer-controlled amplifier (MultiClamp 700B; Axon Instruments, Union City, Calif.), digitized (Digidata 1440; Axon Instruments), and acquired with Axoscope 10.1 (Axon Instruments) at a sampling rate of 10 KHz. The liquid junction potential was not corrected and electrode potentials were adjusted to zero before obtaining the whole-cell configuration.

Nucleus accumbens medium spiny neuron synaptic responses were elicited by local electrical stimulation (0.05 to 0.30 mA square pulses of 0.3 ms duration delivered every 20 s) of excitatory inputs using a bipolar electrode made from a pair of twisted Teflon-coated nichrome wires (tips separated by approximately 200 μm) and placed ˜300 μm lateral to the recorded neurons. The intensity of stimulation was chosen from the minimum amount of current necessary to elicit a synaptic response with <15% variability in amplitude during baseline recording⁵². Only neurons that retained such synaptic response reliability during the subsequent 20 min of baseline recording were included in the present study. If the current intensity required was >0.3 mA, the neuron was discarded.

All recordings were conducted in voltage clamp configuration at 33-35° C. in the absence of TTX. Control and drug-containing aCSF were continuously oxygenated throughout the experiments. After 20-30 min of baseline recording, a solution containing the GluR2-lacking AMPA receptor antagonist Naspm (100-200 μM) was perfused for 10 min followed by a 20-30 min washout period. Changes in input resistance, spontaneous EPSC (frequency and amplitude), evoked EPSC amplitude and paired-pulse ratio (at 50 ms interval) were analyzed before and after drug application. In addition, we collected several points of the current-voltage relationship (holding Vm at −70 mV, −50 mV, −30 mV, +20 mV, +40 mV and +60 mV) of the evoked AMPA-mediated EPSC during baseline to compute the rectification index.

The rectification index was calculated by correcting any potential shifts in the reversal potential values (Eᵣₑᵥ)⁵³ and computed using the following equation: RI=(I₋₇₀/(70−Eᵣₑᵥ))/(I₊₄₀/(40−Eᵣₑᵥ)). Thus, RI is expressed as a ratio that will increase when rectification increases. I₋₇₀ and I₊₄₀ are the EPSC current amplitudes recorded by holding the membrane potential at −70 mV and +40 mV, respectively. The Eᵣₑᵥ values were obtained from the I-V relationship. Finally, we performed frequency and amplitude analyses of spontaneous AMPA receptor-mediated events using Clampfit 10 (Axon Instruments). All comparisons were performed from 3 min segments of baseline recordings acquired at 10 KHz. For each neuron, we assessed cumulative histograms and conducted Kolmogorov Smimov tests. All measures are expressed as mean±S.E.M. All neurons included in the present study were labeled with Neurobiotin. Their location and morphology were further confirmed as medium spiny neurons in the core region of the nucleus accumbens.

### Statistical Analyses

Data from self-administration experiments were analyzed with the statistical program SPSS (GLM procedure). The nose-poke or lever-press data from the extinction tests for cue-induced cocaine-seeking were analyzed with Analysis of Variance (ANOVA) with Withdrawal Day (1, 45) as the between-subjects factor, and Hole or Lever (previously active, inactive) as the within-subject factor. For the Naspm accumbens injection experiment, the statistical analyses also included the within-subjects factor of Session Hour. For biochemical studies, group differences in protein levels were analyzed by ANOVA using Drug exposure (saline, cocaine) or Extinction test (yes, no) and Withdrawal Day (1, 45) as the between-subjects factors, followed by a post hoc Tukey test. For experiments on the effect of Naspm on cocaine or sucrose self-administration, the ANOVA included the within-subjects factors of Naspm Dose (Vehicle, 40 μg) and Session Hour (1-6). For electrophysiological studies, drug effects were compared using Student's t-test or repeated-measures ANOVA. Differences between experimental conditions were considered statistically significant when p<0.05.

### REFERENCES

1. Gawin, F. H. & Kleber, H. D. Abstinence symptomatology and psychiatric diagnosis in cocaine abusers. Clinical observations. Arch. Gen. Psychiatry. 43:107-113 (1986).
2. Grimm, J. W., Hope, B. T., Wise, R. A. & Shaham, Y. Incubation of cocaine craving after withdrawal. Nature. 412:141-142 (2001).
3. Lu, L., Grimm, J. W., Hope, B. T. & Shaham, Y. Incubation of cocaine craving after withdrawal: a review of preclinical data. Neuropharmacology. 47 Suppl 1:214-226 (2004).
4. Neisewander, J. L. et al. Fos protein expression and cocaine-seeking behavior in rats after exposure to a cocaine self-administration environment. J. Neurosci. 20:798-805 (2000).
5. Cornish, J. L. & Kalivas, P. W. Glutamate transmission in the nucleus accumbens mediates relapse in cocaine addiction. J. Neurosci. 20:RC89 (2000).
6. Di Clano, P., Cardinal, R. N., Cowell, R. A., Little, S. J. & Everitt, B. J. Differential involvement of NMDA, AMPA/kainate, and dopamine receptors in the nucleus accumbens core in the acquisition and performance of pavlovian approach behavior. J. Neurosci. 21:9471-9477 (2001).
7. Suto, N. et al. Previous exposure to psychostimulants enhances the reinstatement of cocaine seeking by nucleus accumbens AMPA. Neuropsychopharmacology. 29:2149-2159 (2004).
8. Cull-Candy, S., Kelly, L. & Farrant, M. Regulation of Ca2+-permeable AMPA receptors: synaptic plasticity and beyond. Curr. Opin. Neurobiol. 16:288-297 (2006).
9. Liu, S. J. & Zukin, R. S. Ca2+-permeable AMPA receptors in synaptic plasticity and neuronal death. Trends Neurosci. 30:126-134 (2007).
10. Kalivas, P. W. & Volkow, N. D. The neural basis of addiction: a pathology of motivation and choice. Am. J. Psychiatry. 162:1403-1413 (2005).
11. Kauer, J. A. & Malenka, R. C. Synaptic plasticity and addiction. Nat. Rev. Neurosci. 8:844-858 (2007).
12. Wolf, M. E. The role of excitatory amino acids in behavioral sensitization to psychomotor stimulants. Prog. Neurobiol. 54:679-720 (1998).
13. Boudreau, A. C., Reimers, J. M., Milovanovic, M. & Wolf, M. E. Cell surface AMPA receptors in the rat nucleus accumbens increase during cocaine withdrawal but internalize after cocaine challenge in association with altered activation of mitogen-activated protein kinases. J. Neurosci. 27:10621-10635 (2007).
14. Boudreau, A. C. & Wolf, M. E. Behavioral sensitization to cocaine is associated with increased AMPA receptor surface expression in the nucleus accumbens. J. Neurosci. 25:9144-9151 (2005).
15. Meredith, G. E. The synaptic framework for chemical signaling in nucleus accumbens. Annals of the New York Academy of Sciences. 877:140-156 (1999).
16. Everitt, B. J. & Robbins, T. W. Neural systems of reinforcement for drug addiction: from actions to habits to compulsion. Nat. Neurosci. 8:1481-1489 (2005).
17. Robinson, T. E. & Kolb, B. Structural plasticity associated with exposure to drugs of abuse. Neuropharmacology. 47 Suppl 1:33-46 (2004).
18. Goto, Y. & Grace, A. A. Dopamine-dependent interactions between limbic and prefrontal cortical plasticity in the nucleus accumbens: disruption by cocaine sensitization. Neuron. 47:255-266 (2005).
19. Kourrich, S., Rothwell, P. E., Klug, J. R. & Thomas, M. J. Cocaine experience controls bidirectional synaptic plasticity in the nucleus accumbens. J. Neurosci. 27:7921-7928 (2007).
20. Pierce, R. C., Bell, K., Duffy, P. & Kalivas, P. W. Repeated cocaine augments excitatory amino acid transmission in the nucleus accumbens only in rats having developed behavioral sensitization. J. Neurosci. 16:1550-1560 (1996).
21. Yao, W. D. et al. Identification of PSD-95 as a regulator of dopamine-mediated synaptic and behavioral plasticity. Neuron. 41:625-638 (2004).
22. Hollander, J. A. & Carelli, R. M. Cocaine-associated stimuli increase cocaine seeking and activate accumbens core neurons after abstinence. J. Neurosci. 27:3535-3539 (2007).
23. Mead, A. N., Zamanillo, D., Becker, N. & Stephens, D. N. AMPA-receptor GluR1 subunits are involved in the control over behavior by cocaine-paired cues. Neuropsychopharmacology. 32:343-353 (2007).
24. Sutton, M. A. et al. Extinction-induced upregulation in AMPA receptors reduces cocaine-seeking behaviour. Nature. 421:70-75 (2003).
25. Beveridge, T. J., Smith, H. R., Daunais, J. B., Nader, M. A. & Porrino, L. J. Chronic cocaine self-administration is associated with altered functional activity in the temporal lobes of non human primates. Eur. J. Neurosci. 23:3109-3118 (2006).
26. Ju, W. et al. Activity-dependent regulation of dendritic synthesis and trafficking of AMPA receptors. Nat. Neurosci. 7:244-253 (2004).
27. O'Brien, R. J. et al. Activity-dependent modulation of synaptic AMPA receptor accumulation. Neuron. 21:1067-1078 (1998).
28. Bellone, C. & Luscher, C. Cocaine triggered AMPA receptor redistribution is reversed in vivo by mGluR-dependent long-term depression. Nat. Neurosci. 9:636-641 (2006).
29. Mameli, M., Balland, B., Lujan, R. & Luscher, C. Rapid synthesis and synaptic insertion of GluR2 for mGluR-LTD in the ventral tegmental area. Science (New York, N.Y. 317:530-533 (2007).
30. Tseng, K. Y. & O'Donnell, P. Dopamine-glutamate interactions controlling prefrontal cortical pyramidal cell excitability involve multiple signaling mechanisms. J. Neurosci. 24:5131-5139 (2004).
31. Paxinos, G. & Watson, C. The rat brain in stereotaxic coordinates. Elsevier Academic Press, Amsterdam (2005).
32. Bossert, J. M., Poles, G., Wihbey, K., Koya, U. & Shaham, Y. Differential effects of blockade of dopamine D1-family receptors in nucleus accumbens core or shell on reinstatement of heroin seeking induced by contextual and discrete cues. J. Neurosci. 27, 12655-1266 (2007)
33. Lu, L., Uejima, J. L., Gray, S. M., Bossert, J. M. & Shaham, Y. Systemic and central amygdala injections of the mGluR_{2/3} agonist LY379268 attenuate the expression of incubation of cocaine craving. Biol. Psych. 61, 591-598 (2007).
34. Takazawa, A. et al. Potent and long-lasting anticonvulsant effects of 1-naphthylacetyl spermine, an analogue of Joro spider toxin, against amygdaloid kindled seizures in rats. Brain Res. 706, 173-176 (1996)
35. Noh, K. M. et al. Blockade of calcium-permeable AMPA receptors protects hippocampal neurons against global ischemia-induced death. Proc. Natl. Acad. Sci. U.S.A. 102, 12230-12235 (2005).
36. Boudreau, A. C. & Wolf, M. E. Behavioral sensitization to cocaine is associated with increased AMPA receptor surface expression in the nucleus accumbens. J. Neurosci. 25, 9144-9151 (2005).
37. Boudreau, A. C., Reimers, J. M., Milovanovic, M. & Wolf, M. E. Cell surface AMPA receptors in the rat nucleus accumbens increase during cocaine withdrawal but internalize after cocaine challenge in association with altered activation of mitogen-activated protein kinases. J. Neurosci. 27, 10621-10635 (2007).
38. Hall, R. A., Hansen, A., Andersen, P. H. & Soderling, T. R. Surface expression of the AMPA receptor subunits GluR1, GluR2, and GluR4 in stably transfected baby hamster kidney cells. J. Neurochem. 68, 625-630 (1997).
39. Hall, R. A. & Soderling, T. R. Differential surface expression and phosphorylation of the N-methyl-D-aspartate receptor subunits NR1 and NR2 in cultured hippocampal neurons. J. Biol. Chem. 272, 4135-4140 (1997).
40. Hall, R. A. & Soderling, T. R. Quantitation of AMPA receptor surface expression in cultured hippocampal neurons. Neurosci. 78, 361-371 (1997).
41. Archibald, K., Perry, M. J., Molnar, E. & Henley, J. M. Surface expression and metabolic half-life of AMPA receptors in cultured rat cerebellar granule cells. Neuropharmacol. 37, 1345-1353 (1998).
42. Broutman, G. & Baudry, M. Involvement of the secretory pathway for AMPA receptors in NMDA-induced potentiation in hippocampus. J. Neurosci. 21, 27-34 (2001).
43. Clayton, D. A., Grosshans, D. R. & Browning, M.D. Aging and surface expression of hippocampal NMDA receptors. J. Biol. Chem. 277, 14367-14369 (2002).
44. Grosshans, D. R., Clayton, D. A., Coultrap, S. J. & Browning, M.D. LTP leads to rapid surface expression of NMDA but not AMPA receptors in adult rat CA1. Nat. Neurosci. 5, 27-33 (2002).
45. Grosshans, D. R., Clayton, D. A., Coultrap, S. J. & Browning, M.D. Analysis of glutamate receptor surface expression in acute hippocampal slices. Sci STKE 137 (2002).
46. Gerges, N. Z. et al. Independent functions of hsp90 in neurotransmitter release and in the continuous synaptic cycling of AMPA receptors. J. Neurosci. 24, 4758-4766 (2004).
47. Sans, N. et al. Aberrant formation of glutamate receptor complexes in hippocampal neurons of mice lacking the GluR2 AMPA receptor subunit. J. Neurosci. 23, 9367-9373 (2003).
48. Wenthold, R. J., Petralia, R. S., Blahos, J. II & Niedzielski, A. S. Evidence for multiple AMPA receptor complexes in hippocampal CA1/CA2 neurons. J. Neurosci. 16, 1982-1989 (1996).
49. Bernard, V., Somogyi, P. & Bolam, J. P. Cellular, subcellular, and subsynaptic distribution of AMPA-type glutamate receptor subunits in the neostriatum of the rat. J. Neurosci. 17, 819-833.
50. Stefani, A., Chen, Q., Flores-Hernandez, J., Jiao, Y., Reiner, A. & Surmeier, D. J. Physiological and molecular properties of AMPA/Kainate receptors expressed by striatal medium spiny neurons. Dev. Neurosci. 20, 242-252.
51. Tseng, K. Y. & O'Donnell, P. Dopamine-glutamate interactions controlling prefrontal cortical pyramidal cell excitability involve multiple signaling mechanisms. J. Neurosci. 24, 5131-5139 (2004).
52. Tseng, K. Y. & O'Donnell, P. D2 dopamine receptors recruit a GABA component for their attenuation of excitatory synaptic transmission in the adult rat prefrontal cortex. Synapse 61, 843-850 (2007).
53. Balland, B., Lachamp, P., Strube, C., Kessler, J. P. & Tell, F. Glutamatergic synapses in the rat nucleus tractus solitarii develop by direct insertion of calcium-impermeable AMPA receptors and without activation of NMDA receptors. J. Physiol. 574, 245-61 (2006).

From the foregoing, it will be observed that numerous variations and modifications may be effected without departing from the spirit and scope of the invention. It is to be understood that no limitation with respect to the specific apparatus illustrated herein is intended or should be inferred. It is, of course, intended to cover by the appended claims all such modifications as fall within the scope of the claims.

What is claimed is:

1. A method for ameliorating cue-induced cocaine craving in abstinent cocaine addicts by administering a compound capable of blockade of GluR2-lacking AMPA receptors.

2. A method for ameliorating cue-induced cravings for an addictive substance in abstinent addicts by administering a compound capable of blockade of GluR1-lacking AMPA receptors.

3. The method of claim 2 wherein the substance is a pharmaceutical drug, an illicit drug, alcohol, caffeine and nicotine.
